(19) United States
(12) Patent Application Publication
(10) Pub. No.: US 2019/0094101 A1
(43) Pub. Date: Mar. 28, 2019

(54) LEAK SENSOR ASSEMBLIES AND SYSTEMS UTILIZING SAME

(71) Applicant: Molex, LLC, Lisle, IL (US)

(72) Inventors: Marko SPIEGEL, LaFox, IL (US); Alexander S. CHERNYSHOV, Naperville, IL (US); Wenfeng PENG, North Aurora, IL (US); Reichl B. HASKELL, Glen Ellyn, IL (US); David R. MASSNER, Joliet, IL (US); Dumitru ROSCA, Lisle, IL (US)

(73) Assignee: Molex, LLC, Lisle, IL (US)

(21) Appl. No.: 16/087,185

(22) PCT Filed: Mar. 20, 2017
(86) PCT No.: PCT/US2017/023213

§ 371 Date: Sep. 21, 2018

Related U.S. Application Data
(60) Provisional application No. 62/311,093, filed on Mar. 21, 2016.

(51) Int. Cl. G01M 3/28

(52) U.S. Cl. CPC G01M 3/28; G01N 33/0047; G01M 3/2853; G01M 3/2876

(57) ABSTRACT

The present disclosure provides a leak sensor assembly which is configured to detect the leakage of gas from a component of a piping system. The leak sensor assembly includes a housing and at least one sensor. The housing is configured to be positioned proximate to the component and forms a pocket which allows gas leaked from the component to concentrate within the pocket in a generally uniform dispersion. The at lease one sensor is configured to detect the presence and concentration of gas within the pocket of the housing.

### RELATED APPLICATIONS

This application claims the domestic of U.S. Ser. No. 62/311,093, filed on Mar. 21, 2016, the contents of which are incorporated herein in its entirety.

### TECHNICAL FIELD

This disclosure relates to the field of sensors, more specifically to the field of leak sensor assemblies and systems utilizing same.

### DESCRIPTION OF RELATED ART

Process plants, such as petroleum refineries and chemical manufacturing facilities, have many complex issues associated with gas leaks. In October 2007, the United States Environmental Protection Agency (“EPA”) issued a document entitled “Leak Detection and Repair—A Best Practices Guide”. According to this document, the EPA has determined that leaking equipment, such as valves, pumps, and connectors, are the largest source of emissions of gases, such as volatile organic compounds (“VOCs”) and volatile hazardous air pollutants (“VHAPs”), from petroleum refineries and chemical manufacturing facilities.

VOCs contribute to the formation of ground-level ozone. Ozone is a major component of smog, and may cause or aggravate respiratory disease. Many areas of the United States, particularly those areas where refineries and chemical facilities are located, do not meet the National Ambient Air Quality Standard (“NAAQS”) for ozone.

Some species of VOCs are also classified as VHAPs. Some known or suspected effects of exposure to VHAPs include cancer, reproductive effects, and birth defects. The highest concentrations of VHAPs tend to be closest to the emission source, where the highest public exposure levels are also often detected. Some common VHAPs emitted from refineries and chemical plants include acetaldehyde, benzene, formaldehyde, methylene chloride, naphthalene, toluene, and xylene.

A typical refinery or chemical plant can emit hundreds of tons per year of VOCs from leaking equipment, such as valves, connectors, pumps, sampling connections, compressors, pressure-relief devices, and open-ended lines.

Valves are used to either restrict or allow the movement of fluids. Valves come in numerous varieties and, except for connectors, are the most common piece of process equipment in industry. Leaks from valves usually occur at the stem or gland area of the valve body and are commonly caused by failure of the valve packing or O-ring.

Connectors are components such as flanges and fittings used to join piping and process equipment together. Gaskets and blinds are usually installed between flanges. Leaks from connectors are commonly caused from gasket failures and improperly torqued bolts on flanges.

Pumps are used to move fluids from one point to another. Two types of pumps extensively used in petroleum refineries and chemical plants are centrifugal pumps and positive displacement, or reciprocating pumps. Leaks from pumps typically occur at the seal.

Sampling connections are utilized to obtain samples from within a process. Leaks from sampling connections usually occur at the outlet of the sampling valve when the sampling line is purged to obtain the sample.

Compressors are designed to increase the pressure of a fluid and provide motive force. They can have rotary or reciprocating designs. Leaks from compressors most often occur from the seals.

Pressure-relief devices are safety devices designed to protect equipment from exceeding the maximum allowable working pressure. Pressure relief valves and rupture disks are examples of pressure relief devices. Leaks from pressure relief valves can occur if the valve is not sealed properly, operating too close to the set point, or if the seal is worn or damaged. Leaks from rupture disks can occur around the disk gasket if not properly installed.

Open-ended lines are pipes or hoses open to the atmosphere or surrounding environment. Leaks from open-ended lines occur at the point of the line open to the atmosphere and are usually controlled by using caps, plugs, and flanges. Leaks can also be caused by the incorrect implementation of the block and bleed procedure.

In a typical facility, most of the emissions are from valves and pumps because of moving parts inside. The major cause of emissions is seal or gasket failure due to normal wear or improper maintenance.

Facilities can control emissions from equipment leaks by implementing a leak detection and repair (“LDAR”) program. LDAR is a work practice designed to identify leaking equipment so that emissions can be reduced through repairs. A component that is subject to LDAR requirements must be monitored at specified, regular intervals to determine whether it is leaking. Any leaking component must then be repaired or replaced within a specified period of time.

Alternatively, or in conjunction with a LDAR program, facilities can control emission from equipment leaks by modifying/replacing leading equipment with “leakless” or “seal less” components. Leakless and seal less components can be effective at minimizing or eliminating leaks, but their use may be limited by materials of construction considerations and process operating conditions. Installing leakless and seal less components can also be expensive and time-consuming, possibly even requiring a shut-down of all or a part of a facility.

LDAR programs are required by many federal, state and local requirements/standards/regulations (“Regulations”). Most these Regulations require the implementation of a formal LDAR program using EPA Reference Method 21 (40 CFR Part 60, Appendix A) (“Method 21”). Facilities must also ensure that they are complying with the proper equipment leak Regulations if multiple Regulations apply. Emissions reductions from implementing a WAR program potentially reduce product losses, increase safety for workers and operators, decrease exposure of the surrounding community, reduce emissions fees, and help facilities avoid enforcement actions.

While the requirements among the Regulations vary, all LDAR programs consist of five basic elements, namely: (1) identifying components; (2) leak definition; (3) monitoring components; (4) repairing components; and (5) recordkeeping.

Identifying components generally includes the following requirements: (a) assigning a unique identification (ID) number to each regulated component; (b) recording each regulated component and its unique ID number in a log; (c) physically locating each regulated component in the facility, verifying its location on the piping and instrumentation diagrams or process flow diagrams, and updating the log if necessary (some states require a physical tag on each component subject to the LDAR requirements); (d) identifying each regulated component on a site plot plan or on a continuously updated equipment log; and (e) promptly noting in the equipment log when new and replacement pieces of equipment are added and equipment is taken out of service.

As for leak definition, Method 21 requires VOC emissions from regulated components to be measured in parts per million (“ppm”). A leak is detected whenever the measured concentration exceeds the threshold standard (i.e., leak definition) for the applicable regulation. Leak definitions vary by regulation, component type, service (e.g., light liquid, heavy liquid, gas/vapor), and monitoring interval. Some regulations New Source Performance Standards (“NSPS”)) have a leak definition of 10,000 ppm, while other regulations (e.g., National Emission Standards for Hazardous Air Pollutants (“NESHAP”)) use a 500 ppm or 1,000 ppm leak definition. Many equipment leak regulations also define a leak based on visual inspections and observations (such as fluids dripping, spraying, misting, or clouding from or around components), sound (such as hissing), and smell.

In connection with monitoring components, for many Regulations with leak detection provisions, the primary method for monitoring to detect leaking components is Method 21. Method 21 is a procedure used to detect VOC leaks from process equipment using a portable detecting instrument. Monitoring intervals vary according to the applicable regulation, but are typically weekly, monthly, quarterly, and yearly. The monitoring interval generally depends on the component type and periodic leak rate for the component type. In general, Method 21 requires three steps: (1) evaluating instrument performance; (2) calibrating instrument; and (3) monitoring individual components.

Evaluating instrument performance includes: (a) for each VOC measured, the response factor should be <10 unless specified in the applicable regulation. Response factor is the ratio of the known concentration of a VOC compound to the observed meter reading when measured using an instrument calibrated with the reference compound specified in the applicable regulation; (b) the calibration precision should be <10 percent of the calibration gas value. Calibration precision is the degree of agreement between measurements of the same known value, expressed as the relative percentage of the average difference between the meter readings and the known concentration to the known concentration; and (c) the response time should be ≤30 seconds. Response time is the time interval from a step change in VOC concentration at the input of the sampling system to the time at which 90% of the corresponding final value is reached as displayed on the instrument readout meter.

Calibrating instrument includes performing the following before each monitoring episode: (a) letting the instrument warm up; (b) introducing the calibration gas into the instrument probe; and (c) adjusting the instrument meter readout to match the calibration gas concentration value.

Monitoring individual components includes: (a) placing the probe at the surface of the component interface where leakage could occur; (b) moving the probe along the interface periphery while observing the instrument readout; (c) locating the maximum reading by moving the probe around the interface; (d) keeping the probe at the location of the maximum reading for two times the response factor; and (e) if the concentration reading on the instrument readout is above the applicable leak definition, then the component is leaking and must be repaired.

Repairing components generally includes the following requirements: (a) repairing leaking components as soon as practicable, but not later than a specified number of calendar days after the leak is detected; (b) if the repair of any component is technically infeasible without a process unit shutdown, the component may be placed on the Delay of Repair list, the ID number is recorded, and an explanation of why the component cannot be repaired immediately is provided. An estimated date for repairing the component must be included in the facility records; and (c) the component is considered to be repaired only after it has been monitored and shown not to be leaking above the applicable leak definition.

Recordkeeping, for each regulated process, includes: (a) maintaining a list of all ID numbers for all equipment subject to an equipment leak regulation; (b) for valves designated as “unsafe to monitor,” maintaining a list of ID numbers and an explanation/review of conditions for the designation; (c) maintaining detailed schematics, equipment design specifications (including dates and descriptions of any changes), and piping and instrumentation diagrams; and (d) maintaining the results of performance testing and leak detection monitoring, including leak monitoring results per the leak frequency, monitoring leakless equipment, and non-periodic event monitoring.

Recordkeeping, for leaking equipment, includes: (a) attaching ID tags to the equipment; (b) maintaining records of the equipment ID number, the instrument and operator ID numbers, and the date the leak was detected; (c) maintaining a list of the dates of each repair attempt and an explanation of the attempted repair method; (d) noting the dates of successful repairs; and (e) including the results of monitoring tests to determine if the repair was successful.

Attempts have been made to make improvements in LDAR programs. One specific area of desired improvement is in connection with the monitoring of individual components. A single process plant can have hundreds of thousands of components, each of which must be monitored for leakage. While the manual use of a probe is an effective way of monitoring the components for leaks, the manual use has several disadvantages associated with it. More specifically, the manual use of a probe is typically performed by a large pool of skilled labor and/or engineers and, thus, tends to have high associated costs to execute, especially as the manual use of a probe to monitor hundreds of thousands of components can be rather time consuming.

In an effort to promote efficiency and reduce costs, gas cameras based on infrared absorption have been developed to quickly scan components. Examples are FLIR model GF300 & 320, and Opgal model EyeCGas. These infrared cameras are fast in identifying locations of large leaks, but they do not provide quantitative results and they do not offer the same level of sensitivity as conventional Method 21 instruments. Therefore, the EPA approved the use of gas imaging devices as an alternative work practice (AWP) but still requires refineries to perform inspections using a “sniffer” type instrument.

Some refineries and chemical plants may have fixed gas detection systems to monitor certain areas. These systems, however, suffer from outdoor environmental conditions, such as wind, rain, and temperature variations. For example, the effect of wind at a leaking valve is illustrated in FIG. 1. FIG. 1 was generated using ANSYS finite element analysis (FEA) simulations. Depending on the position of the sensor on the valve with respect to the point where the leak is occurring, the sensor's reading may vary from hundreds of parts per million (ppm) down to a few parts per billion (ppb) levels. In order to detect and quantify the leak, the sensor must be placed exactly at the leak location, but the odds of this occurring are not likely and, furthermore, those odds are not odds that are acceptable to the process plants and/or the EPA. Since the negatives associated with using sensors have not been overcome, the tried-and-true method of manual monitoring with an expensive handheld device to reliably locate and quantify the leak continue to be used.

As a result of the foregoing, certain individuals would appreciate further improvements in LDAR programs, including the use of leak detection sensors and assemblies, and systems utilizing same.

### SUMMARY

In an embodiment, the present disclosure provides a leak sensor assembly which is configured to detect the leakage of gas from a component of a piping system. The leak sensor assembly includes a housing and at least one sensor. The housing is configured to be positioned proximate to the component and forms a pocket which allows gas leaked from the component to concentrate within the pocket in a generally uniform dispersion. The at least one sensor is configured to detect the presence and concentration of gas within the pocket of the housing.

### BRIEF DESCRIPTION OF THE DRAWINGS

The present disclosure is illustrated by way of example and not limited in the accompanying figures in which like reference numerals indicate similar elements and in which:

FIG. 1 illustrates the effect of wind on a leak at a point on a valve using ANSYS finite element analysis (FEA) simulations;

FIG. 2A is a perspective view of a first part of a prior art piping system;

FIG. 2B is a perspective view of a body of the first part of the prior art piping system;

FIG. 3A is a perspective view of a second part of a prior art piping system;

FIG. 3B is a perspective view of a connector of the second part of the prior art piping system;

FIG. 4 is a perspective view of a third part of a prior art piping system;

FIG. 5 is a block diagram showing a sensor of the leak sensor assembly in communication with a control unit;

FIG. 6 is a block diagram showing a sensor of the leak sensor assembly as part of a sensor network;

FIG. 7 is a perspective view of a first embodiment of a leak sensor assembly attached to the piping system;

FIGS. 8 and 9 are perspective views of a housing of the leak sensor assembly of FIG. 7;

FIG. 10 is a cross-sectional view of the leak sensor assembly of FIG. 7 attached to the piping system;

FIGS. 11 and 12 are perspective views of a second embodiment of a leak sensor assembly attached to the piping system;

FIG. 13 is a plan view of the leak sensor assembly of FIGS. 11 and 12;

FIG. 14 is an exploded perspective view of a housing of the leak sensor assembly of FIGS. 11 and 12;

FIG. 15 is a perspective view of a half of the housing of FIG. 14;

FIG. 16 is a side elevation view of the leak sensor assembly of FIGS. 11 and 12 attached to the piping system and with a half of the housing removed;

FIG. 17 is a perspective view of a third embodiment of a leak sensor assembly attached to the piping system;

FIGS. 18 and 19 are perspective views of a housing of the leak sensor assembly of FIG. 17;

FIG. 20 is a perspective view of the housing of FIGS. 18 and 19 showing the housing in two halves, but joined together along a line;

FIG. 21 is a perspective view of the housing of FIGS. 18 and 19 showing the housing in two separate halves;

FIG. 22 is a perspective view of a fourth embodiment of a leak sensor assembly attached to the piping system;

FIG. 23 is cross-sectional view of a modified leak sensor assembly of FIG. 22 attached to the piping system;

FIG. 24 is a perspective view showing a filler within the housing of FIGS. 8 and 9;

FIGS. 25-28 are perspective views of the leak sensor assemblies showing a drain opening;

FIG. 29 is a perspective view showing the housing of FIGS. 18-21 with a collar assembly exploded therefrom which is used to releasably attach the sensor to the housing;

FIG. 30 is a perspective view showing the housing of FIGS. 18-21 with the collar assembly of FIG. 29 partially attached thereto;

FIGS. 31 and 32 are perspective views showing the housing of FIGS. 18-21 with the collar assembly attached thereto and showing the sensor;

FIG. 33 is a perspective view of a collar of the collar assembly of FIG. 29;

FIG. 34 is a perspective view of a fifth embodiment of a leak sensor assembly attached to the piping system;

FIG. 35 is a cross-sectional view of a housing of the leak sensor assembly shown in FIG. 34;

FIG. 36 is a perspective view of the housing of the leak sensor assembly shown in FIG. 34;

FIG. 37 is an end view of a housing of the leak sensor assembly shown in FIG. 34;

FIG. 38 is an end view of a housing of the leak sensor assembly shown in FIG. 34 with the sensor partially engaged;

FIG. 39 is a perspective view of a modified fifth embodiment of a leak sensor assembly attached to the piping system;

FIG. 40 is an exploded perspective view of the modified leak sensor assembly of FIG. 38;

FIG. 41 is a partial cross-sectional view of a sixth embodiment of a leak sensor assembly; and

FIG. 42 is a perspective view of a seventh embodiment of a leak sensor assembly.

### DETAILED DESCRIPTION

While the disclosure may be susceptible to embodiment in different forms, there is shown in the drawings, and herein will be described in detail, specific embodiments with the understanding that the present disclosure is to be considered an exemplification of the principles of the disclosure, and is not intended to limit the disclosure to that as illustrated and described herein. Therefore, unless otherwise noted, features disclosed herein may be combined together to form additional combinations that were not otherwise shown for purposes of brevity. It will be further appreciated that in some embodiments, one or more elements illustrated by way of example in a drawing(s) may be eliminated and/or substituted with alternative elements within the scope of the disclosure.

The present disclosure provides embodiments of a leak sensor assembly 100, 200, 300, 400, 500, 600, 700 which can be used to detect leaks occurring at various components of a piping system that are typically found in a process plant, such as valves 20 and connectors 22, 22′ connected to pipes 28, 30, 50. Within a process plant, one or more elaborate piping systems are provided for delivering and managing the flow of liquid and gas materials. These piping systems include a plurality of valves 20 which are configured to control the flow of the material through the pipes 28, 30 of the piping systems, and connectors 22, 22′ which are configured to direct the flow of the material through the pipes 28, 30/pipes 28, 30, 50 within the piping systems. Each valve 20 can be an on/off valve (which either allows for the flow of material past the valve 20 or prevents the flow of material past the valve 20) and/or can be a throttling valve (which controls the flow rate of the material past the valve 20). The valve 20 can be any known type of valve, such as a gate valve or a globe valve.

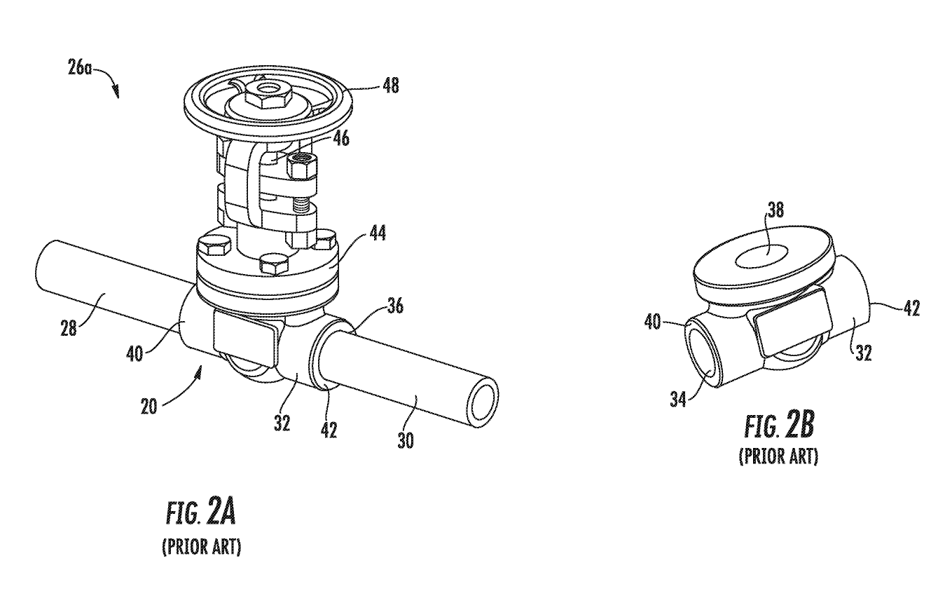

FIGS. 2A and 2B illustrate a first portion 26a of the piping system. The first portion 26a includes a first pipe 28, a second pipe 30, and the valve 20. The valve 20 includes a generally hollow body 32 having first, second and third openings 34, 36, 38 at first and second ends 40, 42 along the length of the valve 20 between the first and second ends 40, 42. The first pipe 28 extends into the first opening 34 and is secured to the first end 40 of the body 32 in a known manner, for example by threading, and the second pipe 30 extends into the second opening 36 and is secured to the second end 42 of the body 32 in a known manner, for example by threading. Thus, a desired material, whether it be a fluid, gas, or slurry, can flow into the body 32 of the valve 20 from the first pipe 28, and thereafter can flow out of the body 32 of the valve 2.0 and into the second pipe 30 for further delivery downstream in the piping system. The valve 20 further includes a bonnet 44 which is configured to close off the opening 38 of the valve 20. The bonnet 44 may be screwed into the body 32 so as to enable any sort of maintenance or repair work to be done without having to remove the entire valve 20 from the piping system. The bonnet 44 further houses other internal parts of the valve 20 to allow for its desired operation. The valve 20 further includes a stem 46 and a handwheel 48. The stem 46 extends downwardly from the handwheel 48 and at least a portion of the stem 46 is housed by the bonnet 44. The handwheel 48 is generally circular in configuration and is used to control the stem 46 which, in turn, controls/prevents/allows the flow of material through the body 32 of the valve 20. Typically, the handwheel 48 is turned clockwise to “close” the valve 20 and counter-clockwise to “open” the valve 20. Thus, a desired material, whether it be a fluid, gas, or slurry, can flow into the body 32 of the valve 20 from the first pipe 28, and thereafter can flow out of the body 32 of the valve 20 and into the second pipe 30 for further delivery downstream in the piping system. The connection between the bonnet 44 and the body 32, and the connection between the bonnet 44 and the stem 46, are understood to be the two most likely places where leaks on the valve 20 will occur. Leaks may also occur between the pipes 28, 30 and the body 32. While the pipes 28, 30 are shown being aligned in FIG. 2A, it is to be understood that the pipes 28, 30 may be an angle relative to each other.

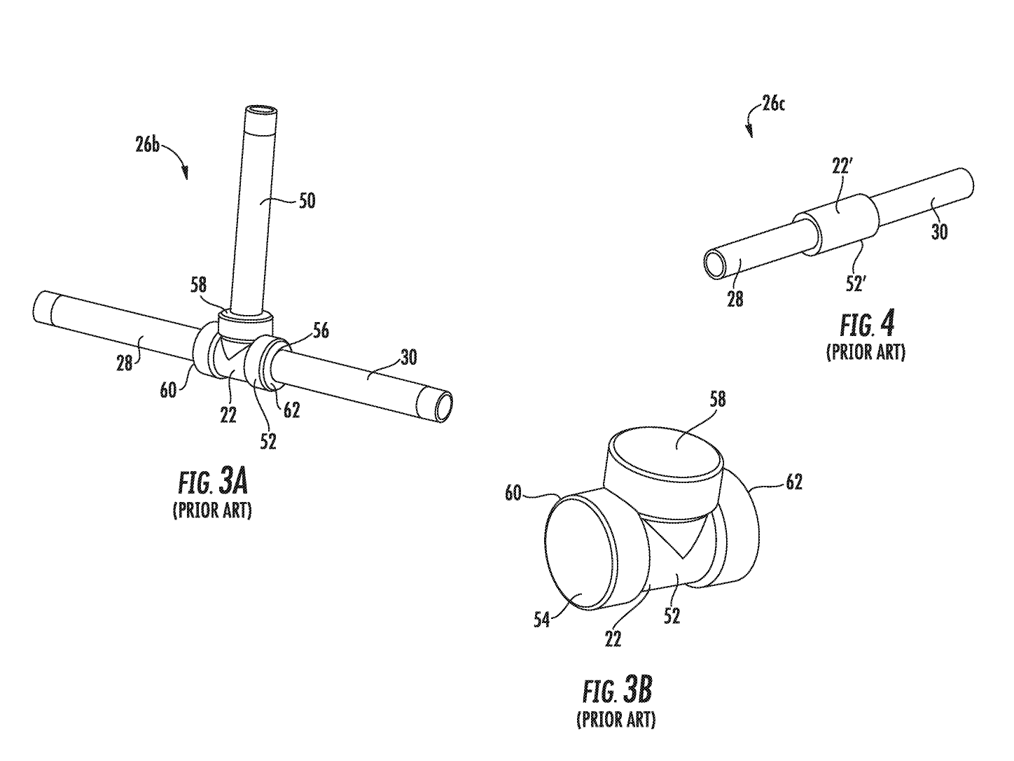

FIGS. 3A and 3B illustrate a second portion 26b of the piping system. The second portion 26b includes a first pipe 28, a second pipe 30, a third pipe 50 and the connector 22. The connector 22 includes a generally hollow body 52 which has openings 54, 56, 58 at its first and second ends 60, 62 along the length of the connector 22 between the first and second ends 60, 62. The first pipe 28 extends into the first opening 54 and is secured to the first end 60 of the body 52 in a known manner, for example by threading; the second pipe 30 extends into the second opening 56 and is secured to the second end 62 of the body 52 in a known manner, for example by threading; and the third pipe 50 extends into the third opening 58 and is secured to the body 52 in a known manner, for example by threading. While the pipes 28, 30 are shown being aligned in FIG. 3A, it is to be understood that the pipes 28, 30 may be an angle relative to each other. While pipe 50 is shown as being perpendicular to the pipes 28, 30 in FIG. 3A, the pipe 50 may be at other angles relative to the pipes 28, 30. Thus, a desired material, whether it be a fluid, gas, or slurry, can flow into the body 52 of the connector 22 from the first pipe 28, and thereafter can flow out of the body 52 of the connector 22 and into the second or third pipes 30, 50 for further delivery downstream in the piping system. The connection between the pipes 28, 30, 50 and the body 52 of the connector 22 provide possible leak paths.

FIG. 4 illustrates a third portion 26c of the piping system. The third portion 26c includes a first pipe 28, a second pipe 30 and the connector 22′. The connector 22′ is identically formed to the connector 22, except that the third opening 58 of the connector 22 is eliminated in connector 22′. Therefore, identical components of the connector 22′ are labeled with the same reference numerals as for the connector 22, but with a prime after the reference numeral. Thus, a desired material, whether it be a fluid, gas, or slurry, can flow into the body 52′ of the connector 22′ from the first pipe 28, and thereafter can flow out of the body 52′ of the connector 22′ and into the second pipe 30 for further delivery downstream in the piping system. The connection between the pipes 28, 30 and the body 52′ of the connector 22′ provide possible leak paths.

While FIGS. 3A-4 show connectors 22, 22′ which are configured to connect two and three pipes together, a connector (not shown) could be provided to connect four pipes together as is known in the art.

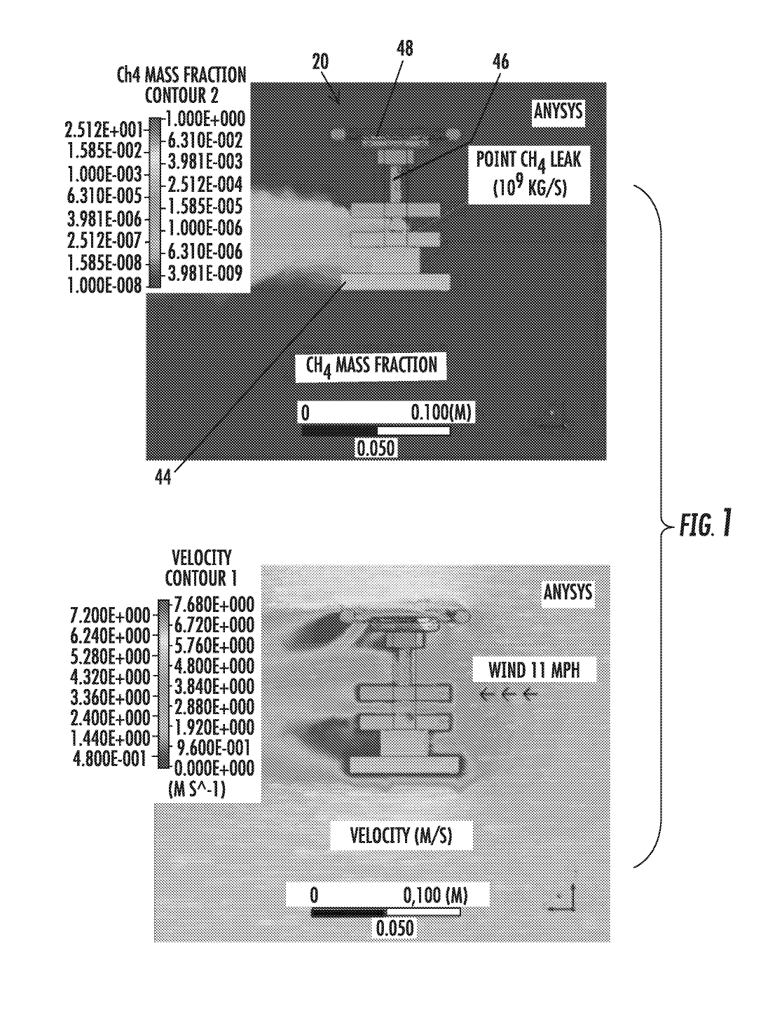

While the portions 26a, 26b, 26c are shown in a particular orientation in FIGS. 1-4, this does not denote a required orientation in use. That is, pipes 28, 30, 50 and valve stem 46 can be at any angle relative to the ground. For example, it appears that the handwheel 48 or the third pipe 50 is vertical in some of the figures; this does not denote a required orientation.

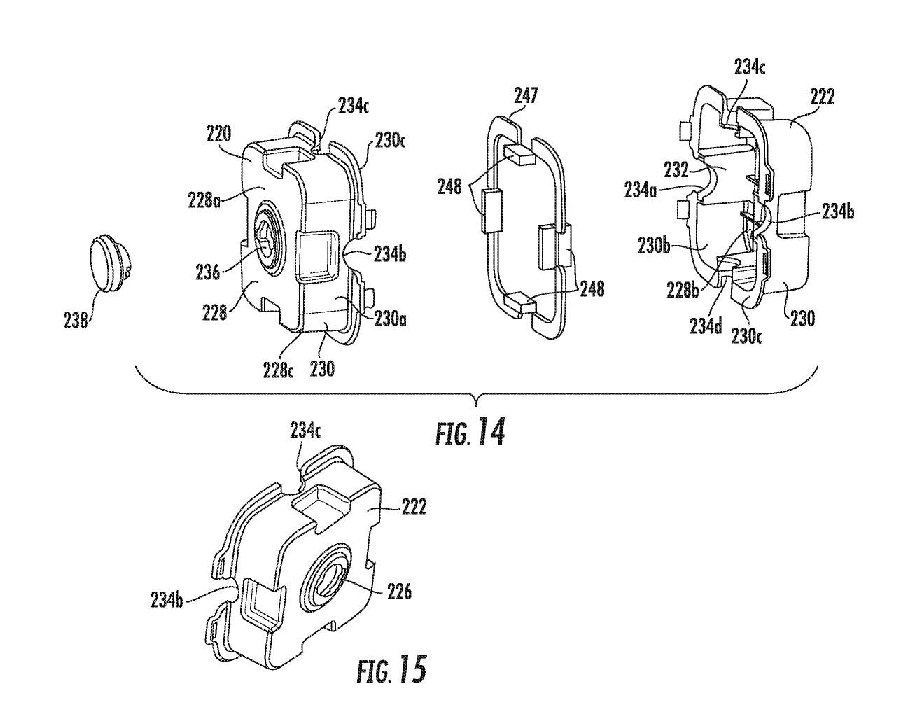

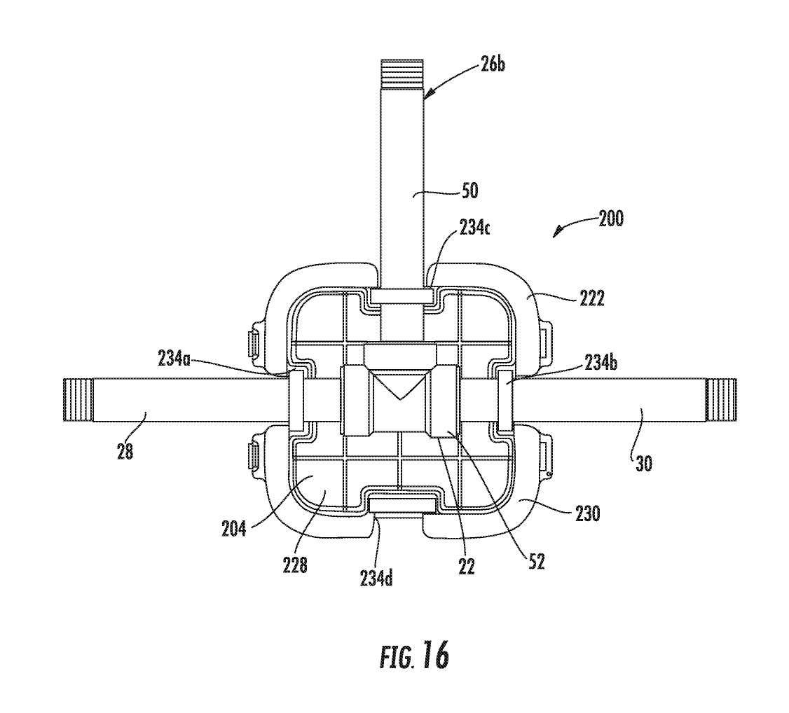

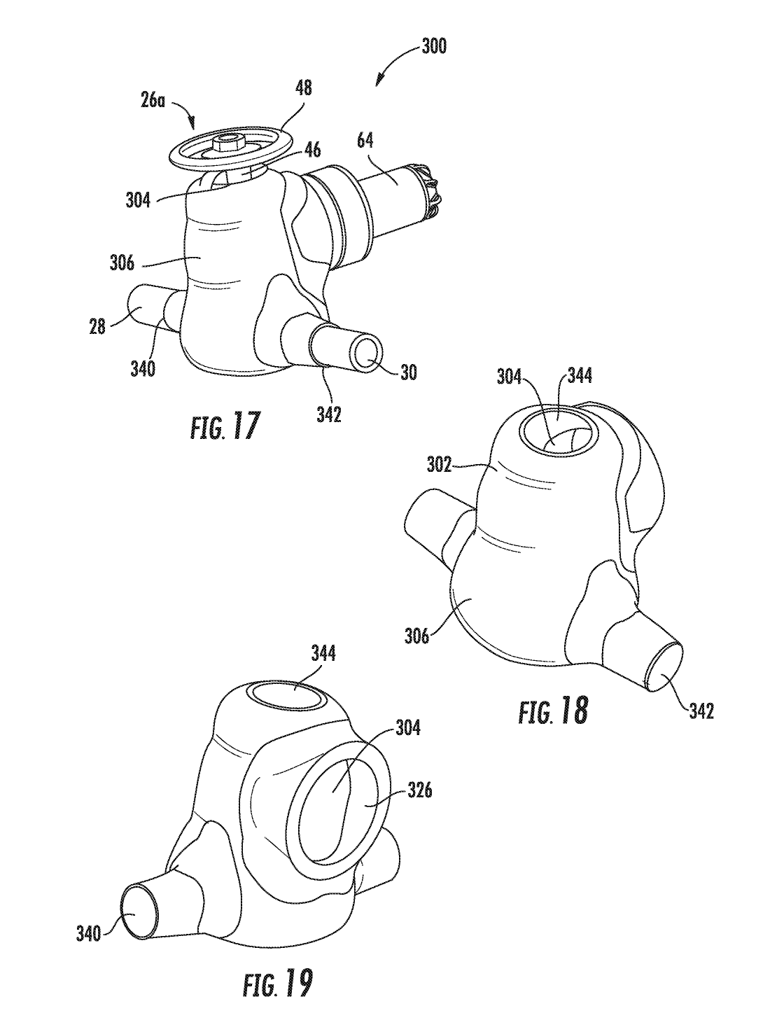

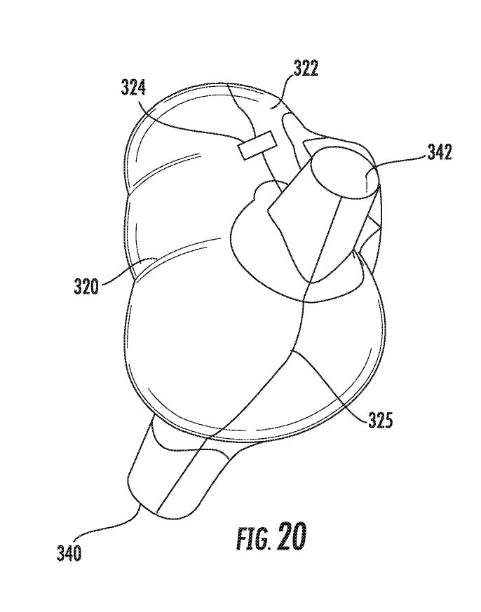

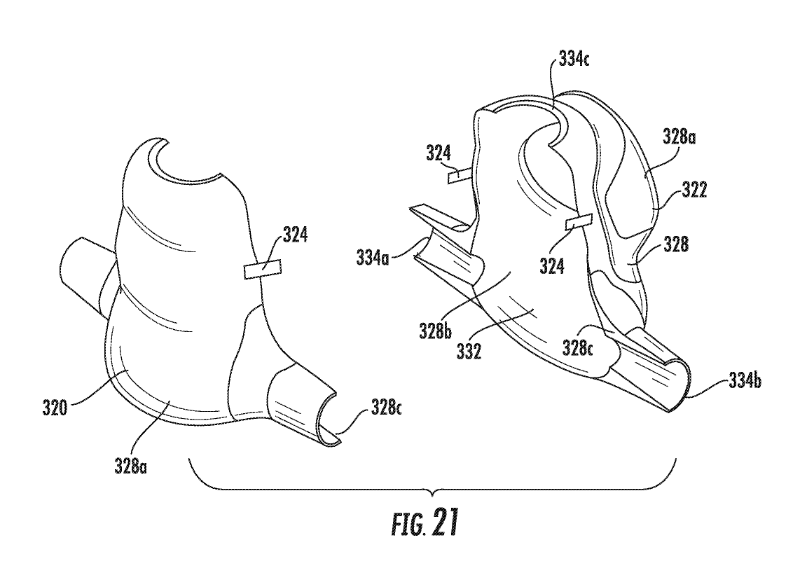

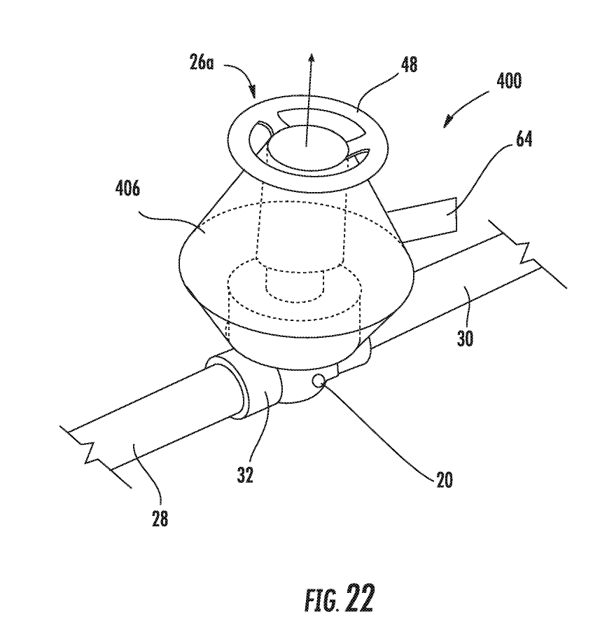

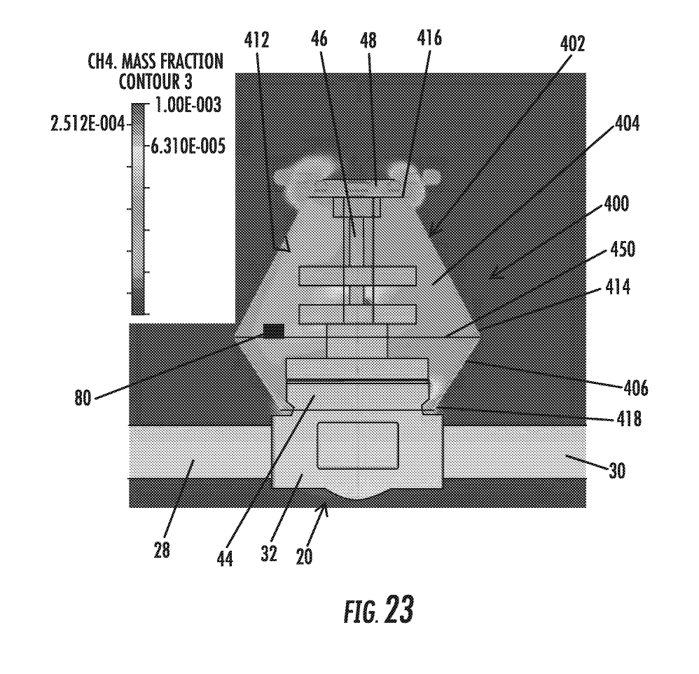

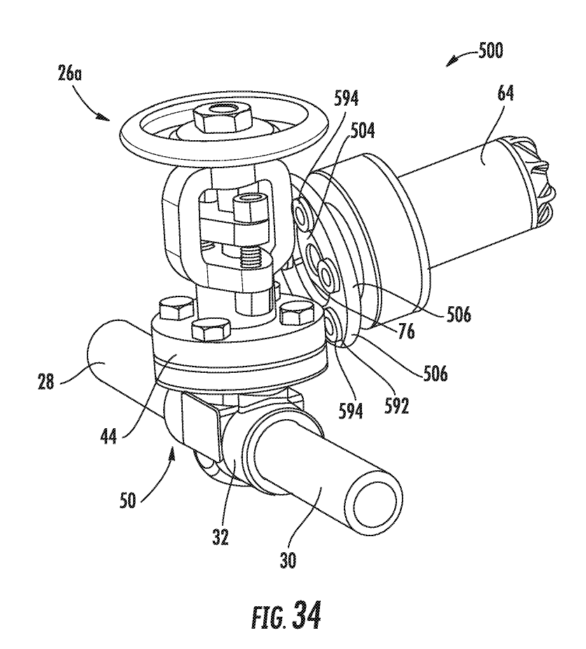

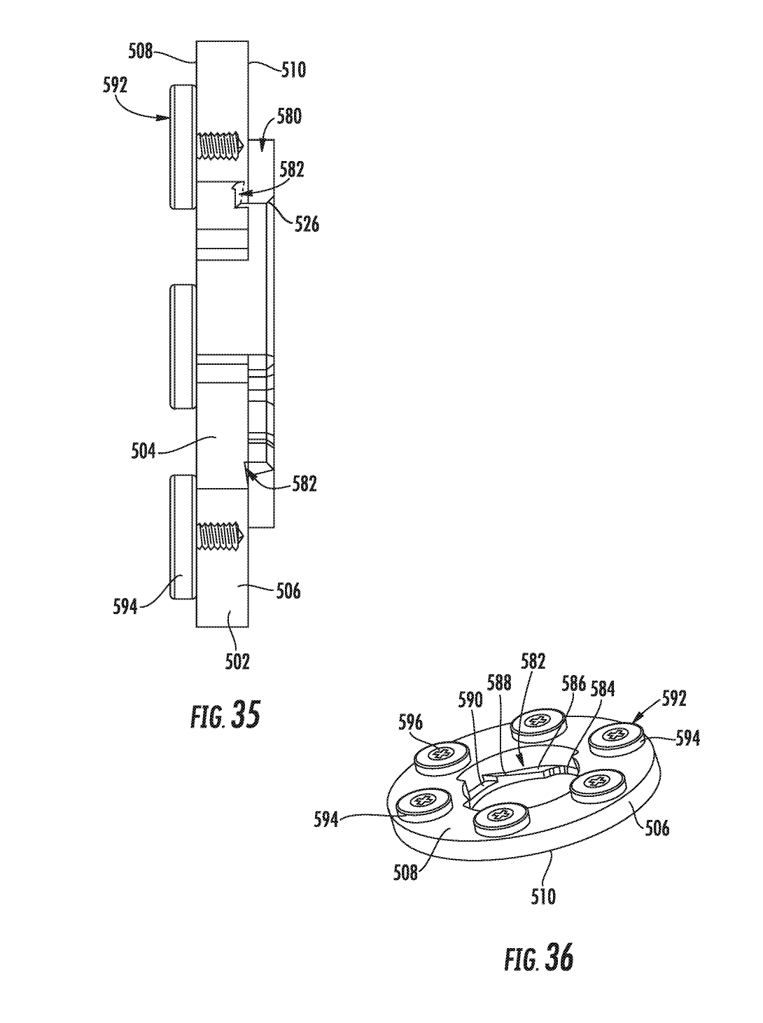

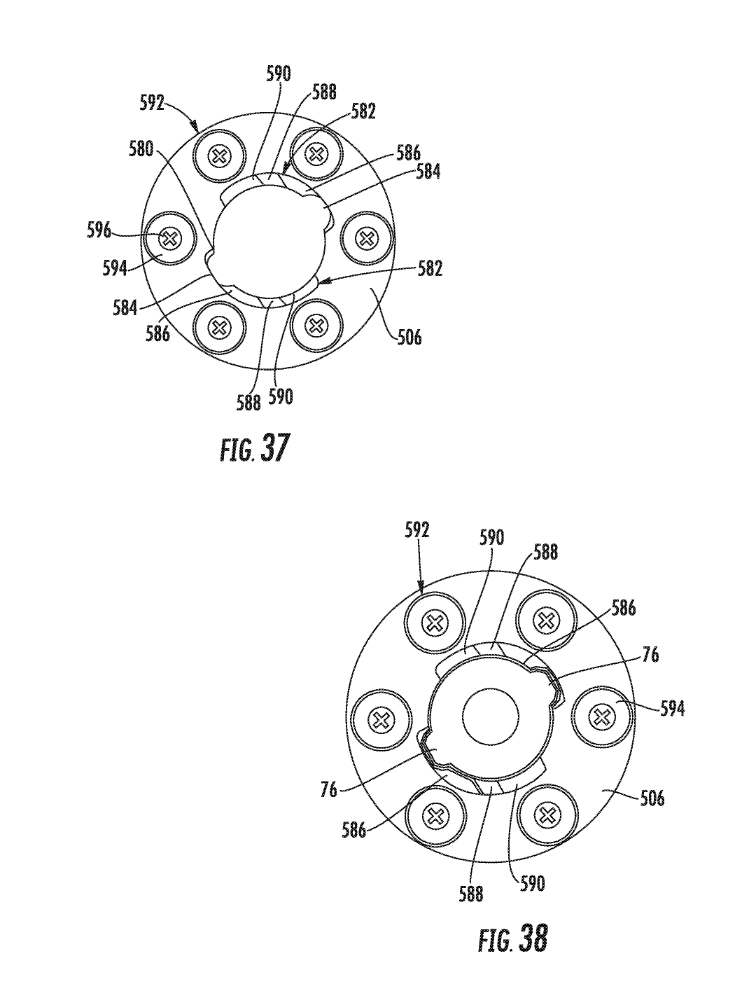

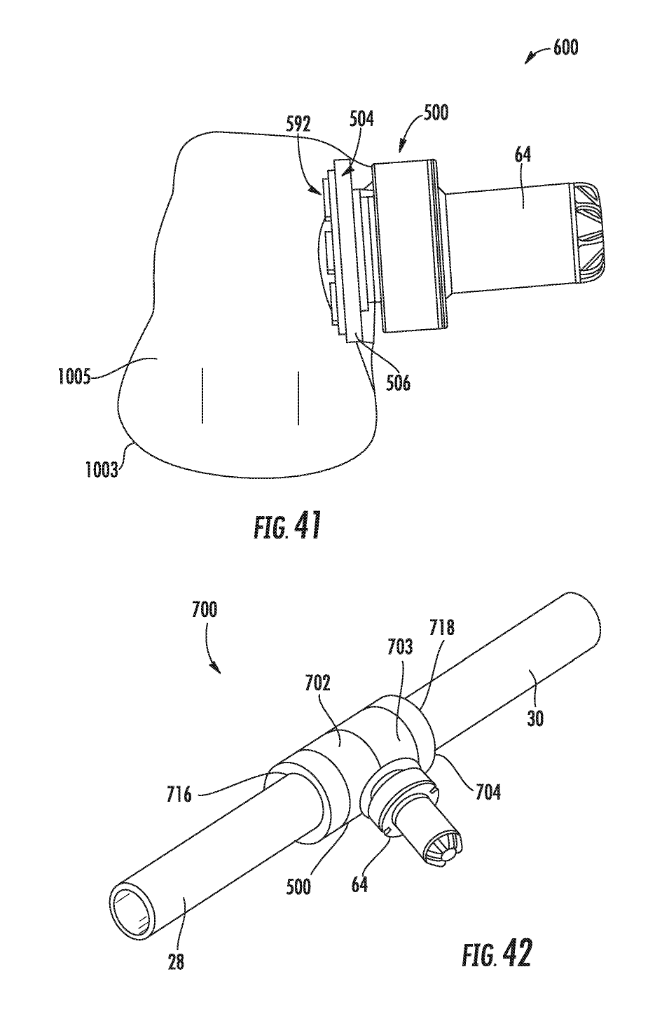

FIGS. 7-10 illustrate a first embodiment of the leak sensor assembly 100. FIGS. 11-16 disclose a second embodiment of the leak sensor assembly 200. FIGS. 17-21 illustrate a third embodiment of the leak sensor assembly 300. FIG. 22 illustrates a fourth embodiment of the leak sensor assembly 400. FIG. 23 illustrates a modification to the fourth embodiment of the leak sensor assembly 400. FIGS. 34-40 illustrate a fifth embodiment of the leak sensor assembly 500. FIG. 41 illustrates a sixth embodiment of the leak sensor assembly 600. FIG. 42 illustrates a seventh embodiment of the leak sensor assembly 700.

Each embodiment of the leak sensor assembly 100, 200, 300, 400, 500, 600, 700 includes a sensor 64 and a housing 102, 202, 302, 402, 502, 702 configured to position the sensor 64 in close proximity to one of the portions 26a, 26b, 26c of the piping system. The housing 102, 202, 302, 402, 502, 702 forms a pocket 104, 204, 304, 404, 504 into which VOCs accumulate so that the sensor 64 can detect the presence and concentration level of the VOCs.

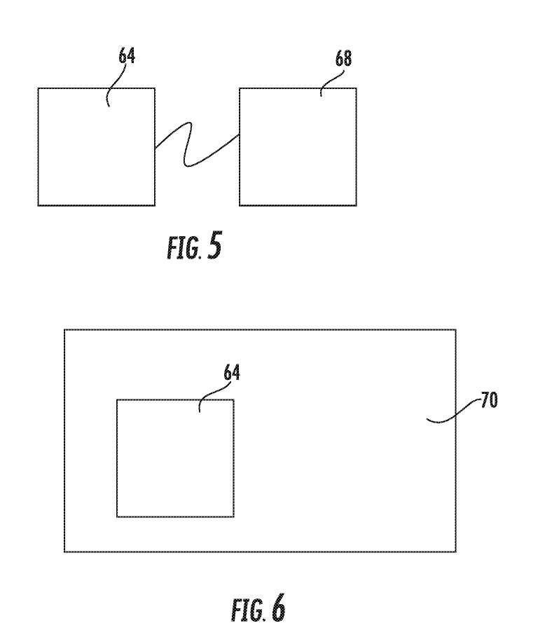

The sensor 64 can be any type of appropriate sensor which is configured to detect volatile organic compounds VOCs, such as infrared, catalytic oxidation, electrochemical, a colorimetric sensor, metal oxide semiconductor (MOS), photoionization detector (PID), a surface acoustic wave sensor, and other chemiresistive sensors. Photoionization detectors, for example, are capable of detecting many VOCs down to sub-ppm level. Electrochemical sensors offer ppm resolution to certain types of VOCs. Infrared sensors are very stable and are particularly suitable for long term, unattended operation. They can detect most VOCs due to characteristic infrared absorption of the hydrocarbon compounds. The sensor 64 could be wired/wirelessly connected to a control unit 68, see FIG. 5, in order to provide information regarding the readings of the sensor 64. The sensor 64 could also be a part of an overall sensor network 70, see FIG. 6, applied across the entire piping system or process plant.

Attention is directed to FIGS. 7-10 and the first embodiment of the leak sensor assembly 100 of the present disclosure. While this embodiment is shown with the first portion 26a of the piping system shown in FIGS. 2A and 2B, this first embodiment can be used with the second or third portions 26b, 26c of the piping system shown in FIGS. 3A, 3B and 4.

The housing 102 is formed from a wall 106 having first and second ends 108, 110, and an inner wall surface 112 and an outer wall surface 114 extending between the ends 108, 110. A first opening 116 is provided through the first end 108 of the wall 106 and a second opening 118 is provided through the second end 110 of the wall 106. The wall 106 defines the pocket 104 therein into which VOCs accumulate when the housing 102 is attached to the piping system as described herein. A sensor opening 126 is provided through the wall 106, and extends from an inner wall surface to the outer wall surface of the wall 106 and is in fluid communication with the pocket 104. The sensor 64 is positioned and held in place within the sensor opening 126 of the housing 102 such that the sensor 64 is configured to detect VOCs within the pocket 104.

In an embodiment, the wall 106 is formed of a rigid and/or semi-rigid material, such as metal, ceramic, thermoplastic (acrylonitrile butadiene styrene (ABS), Noryl, polyethylene, polypropylene, polyvinyl chloride (PVC), polycarbonate, etc.), and particularly high temperature plastics such as liquid crystal polymer (LCP), syndiotactic polystyrene (SPS), polyvinylidene fluoride (PVDF), nylon, and durable high-performance polyimide-based plastics, such as those sold under the registered trademark VESPEL. The material of the wall 106 is also preferably water-proof, weather-proof (e.g., resistant to the formation of cracks, deformation, etc. upon prolonged exposure to the elements), and chemically resistant to organic compounds. The material of the housing 102 can further desirably withstand a wide temperature range, for example, −40° Celsius to 200° Celsius. The housing 102 may be formed of one consistent material or may be formed of layers of materials. The layers of materials may be different.

In an embodiment, the inner wall surface 112 is circular in cross-section, yet conical in configuration from the first opening 116 to the second opening 118. In an embodiment, the outer wall surface 114 matches the configuration of the inner wall surface 112, but the outer wall surface 114 can have any desired configuration, regardless of whether it matches the configuration of the inner wall surface 112.

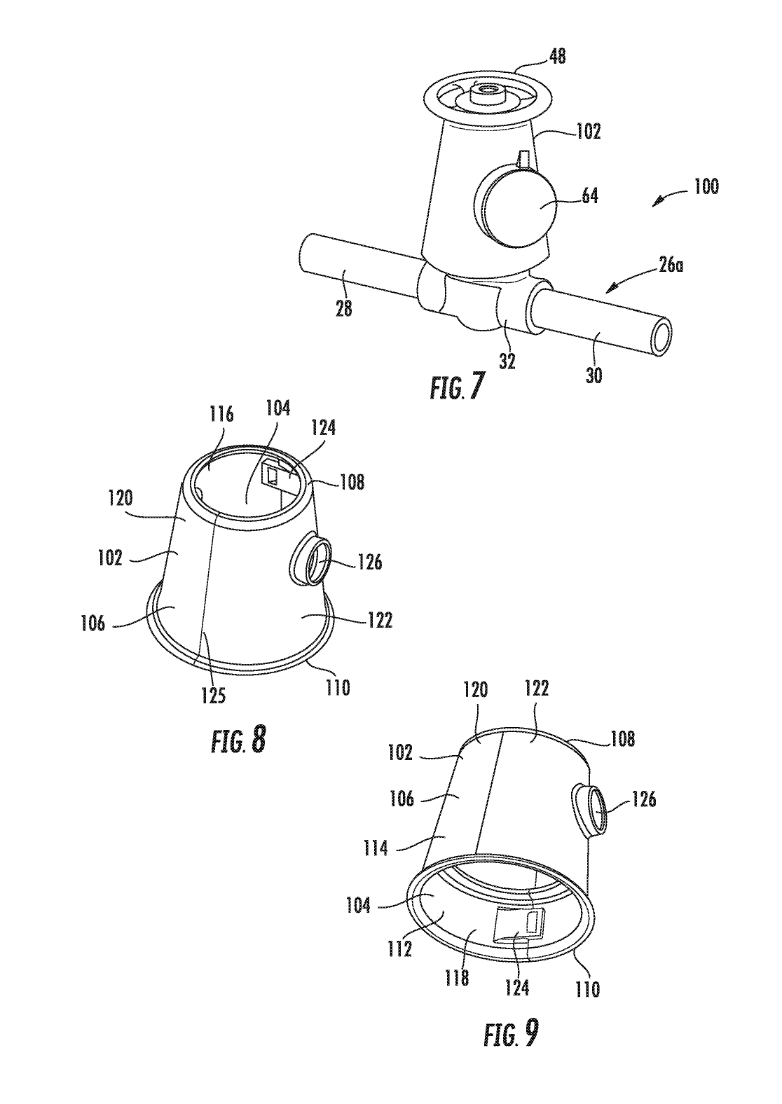

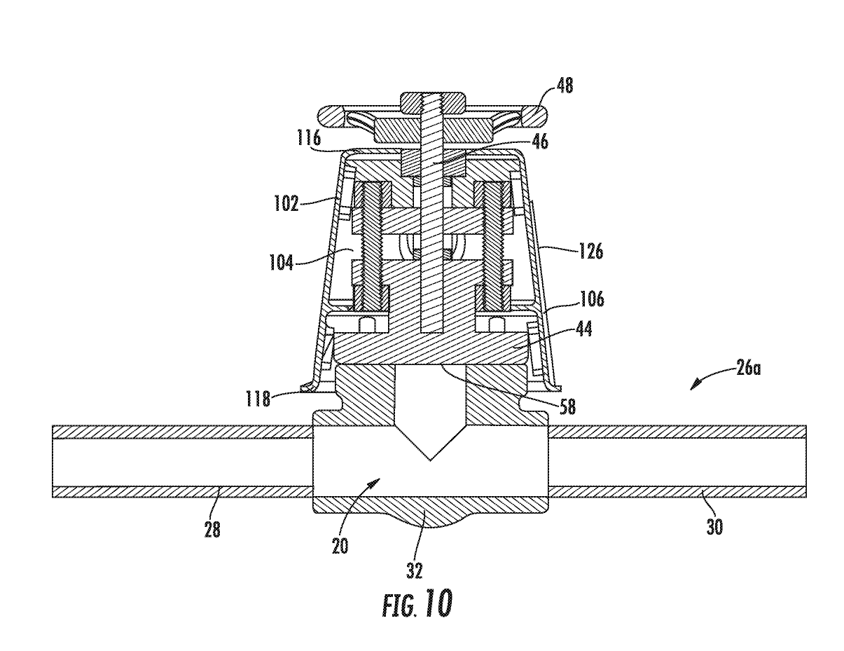

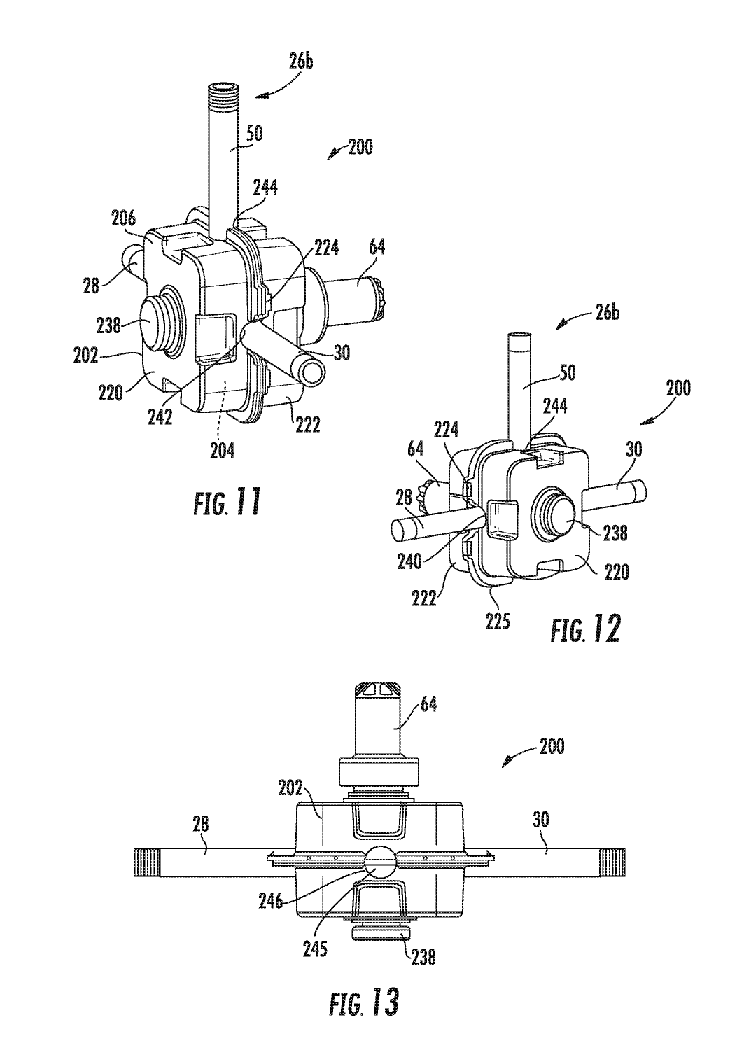

In an embodiment as illustrated in FIGS. 8 and 9, the housing 102 is formed of two halves 120, 122 which are joined together by suitable securing means 124. In an embodiment, each half 120, 122 is shaped as generally a half of a frusto-conical shape such that when the two halves 120, 122 are mated, a frusto-conical shape is formed. The two halves 120, 122 could be hinged/flexible such that the two halves 120, 122 are joined or integrally formed at line 125 which extends from opening 116 to opening 118, but the two halves 120, 122 are separated along the remainder of the intersection between the two halves 120, 122; such the two halves 120, 122 can open and close similar to a clam shell along line 125, and such that when closed the two halves 120, 122 are further joined together by the securing means 124. That is, the housing 102 is formed of a single part which can be opened or closed. The two halves 120, 122 can be completely separate from each other and joined together by the securing means 124. The two halves 120, 122 can be secured to one another using any suitable securing means 124, including, but not limited to, fasteners, latches (as shown), tape, hook-and-loop fasteners (such as those sold under the registered trademark VELCRO), magnets, snaps, buttons, etc. The suitable securing means 124 are also preferably ones that allow for the housing 102 to be removed and reinstalled as desired. In other words, the housing 102 is preferably reusable. In an embodiment, the sensor opening 126 is provided through the wall 106 and extends from the inner wall surface 112 to the outer wall surface 114 and is in fluid communication with the pocket 104. The sensor opening 126 is separated from the first and second openings 116, 118. The sensor opening 126 may be provided at a midpoint of the wall 106 between the first and second openings 116, 118. The sensor opening 126 is sized and positioned based on the sensor 64 to be used and the desired location of the sensor 64. The sensor 64 is configured to be positioned and held in place within the sensor opening 126 of the housing 102 such that the sensor 64 is configured to detect VOCs within the pocket 104.

As shown in FIG. 7, the leak sensor assembly 100 is attached to the first portion 26a of the piping system. The housing 102 is configured to be generally positioned such that a portion of the valve 20 seats within the pocket 104. In an embodiment, the stern 46 seats within the first opening 116 and the bonnet 44 and at least a portion of the body 32 seats within the second opening 118 such that the stern 46, the connection between the stem 46 and the bonnet 44, the bonnet 44, and the connection between the bonnet 44 and the body 32 are generally enclosed by the housing 102. The first opening 116 may be sized to fit loosely around the stem 46 or in close proximity to the stem 46; the second opening 118 may be sized to fit loosely around the body 32 or in close proximity to the body 32.

When the housing 102 is attached to the second portion 26b of the piping system, the housing 102 is configured to be generally positioned such that a portion of one of the pipes 28, 30, 50 and a portion of the connector 22 seats within the pocket 104. In an embodiment, the pipe 28, 30 or 50 seats within the first opening 116 and the body 52 of the connector 22 seats within the second opening 118 such that the connection between the pipe 28, 30 or 50 and the body 52 are generally enclosed by the housing 102. The first opening 116 may be sized to fit loosely around the pipe 28, 30 or 50 or in close proximity to the pipe 28, 30 or 50; the second opening 118 may be sized to fit loosely around the body or in close proximity to the body 52.

When the housing 102 is attached to the third portion 26c of the piping system, the housing 102 is configured to be generally positioned such that the connector 22′ seats within the pocket 104. In an embodiment, the first pipe 28 seats within the first opening 116 and the connector 22′ seats within the pocket 104, and the second pipe 30 seats within the second opening 118 such that the connections between the connector 22′ and the pipes 28, 30 are generally enclosed by the housing 102. The first opening 116 may be sized to fit loosely around the first pipe 28 or in close proximity to the first pipe 28; the second opening 118 may be sized to fit loosely around the second pipe 30 or in close proximity to the second pipe 30. In an embodiment, the first pipe 28 seats within the first opening 116 and the connector 22′ seats within the pocket 104, and the body 52′ of the connector 22′ seats within the second opening 118 such that the connections between the connector 22′ and the pipe 28 are generally enclosed by the housing 102. The first opening 116 may be sized to fit loosely around the first pipe 28 or in close proximity to the first pipe 28; the second opening 118 may be sized to fit loosely around the body 52′ of the connector 22′ or in close proximity to the body 52′ of the connector 22′.

The frusto-conical/conical shape of some embodiments of the inner wall 114 aids in limiting moisture accumulation in the pocket 104 as the moisture runs off. Of course, it is to be understood that while the inner wall 114 is identified as being frusto-conical/conical in shape, the inner wall 114 could take on any other desired shape, or any other shape that is dictated by the configuration of the valve 20 so as to maintain the desired volume of the pocket 104.

Attention is directed to FIGS. 11-16 and a second embodiment of the leak sensor assembly 200 of the present disclosure. While this embodiment is shown with the second portion 26b of the piping system shown in FIGS. 3A and 3B, this second embodiment can be used with the first or third portions 26a, 26c of the piping system shown in FIGS. 2A, 2B and 4.

The housing 202 is formed from a wall 206 having first, second, third and fourth spaced apart openings 240, 242, 244, 246 provided therethrough. In an embodiment, adjacent openings 240, 242, 244, 246 are 90 degrees apart from each other. The wall 206 defines the pocket 204 therein into which VOCs accumulate when the housing 202 is attached to the piping system as described herein. A sensor opening 226 is provided through the wall 206, and extends from an inner wall surface to the outer wall surface of the wall 206 and is in fluid communication with the pocket 204. The sensor 64 is positioned and held in place within the sensor opening 226 of the housing 202 such that the sensor 64 is configured to detect VOCs within the pocket 204.

In an embodiment, the wall 206 is formed of a rigid and/or semi-rigid material, such as metal, ceramic, thermoplastic (acrylonitrile butadiene styrene (ABS), Noryl, polyethylene, polypropylene, polyvinyl chloride (PVC), polycarbonate, etc.), and particularly high temperature plastics such as liquid crystal polymer (LCP), syndiotactic polystyrene (SPS), polyvinylidene fluoride (PVDF), nylon, and durable high-performance polyimide-based plastics, such as those sold under the registered trademark VESPEL. The material of the wall 206 is also preferably water-proof, weather-proof (e.g., resistant to the formation of cracks, deformation, etc. upon prolonged exposure to the elements), and chemically resistant to organic compounds. The material of the housing 202 can further desirably withstand a wide temperature range, for example, −40° Celsius to 200° Celsius. The wall may be formed of one consistent material or may be formed of layers of materials. The layers of materials may be different.

In an embodiment, the housing 202 is formed of two halves 220, 22.2 which are joined together by suitable securing means 224. The two halves 220, 222 could be hinged/flexible such that the two halves 220, 222 are joined or integrally formed at line 225 which extends from opening 240 to opening 242, for example, but the two halves 220, 222 are separated along the remainder of the intersection between the two halves 220, 222; such the two halves 220, 222 can open and close similar to a clam shell along line 225, and such that when closed the two halves 220, 222 are further joined together by the securing means 224. That is, the housing 202 is formed of a single part which can be opened or closed. In an embodiment, when the line 225 is provided, the fourth opening is eliminated. The two halves 220, 222 can be completely separate from each other and joined together by the securing means 224. The two halves 220, 222 can be secured to one another using any suitable securing means 224, including, but not limited to, fasteners, latches (as shown), tape, hook-and-loop fasteners (such as those sold under the registered trademark VELCRO), magnets, snaps, buttons, etc. The suitable securing means 224 are also preferably ones that allow for the housing 202 to be removed and reinstalled as desired. In other words, the housing 202 is preferably reusable.

Each half 220, 222 includes a base wall 228 having a side wall 230 extending outwardly therefrom such that a recess 232 is formed within each half 220, 222. The base wall 228 has an outer wall surface 228a, an inner wall surface 228c and an outer edge 228c. In an embodiment, the side wall 230 extends from the outer edge 228c. The side wall 230 has an outer wall surface 230a, an inner wall surface 230b, and an end surface 230c. The end surface 230c of the side wall 230 forms four half openings 234a, 234b, 234c, 234d which extend toward the base wall 228 and are sized to approximate the shapes of the pipes 28, 30, 50 and/or the stem 46 (and/or the fourth pipe if provided). In an embodiment, the half openings 234a, 234b, 234c, 234d are half circles. In an embodiment, the first half 220 has an opening 236 into which a plug 238 is seated. The opening 236 and plug 238 may be eliminated. In an embodiment, each base wall 228 is square and each side wall 230 is formed of four wall portions which are joined together at corners. In an embodiment, each base wall 228 is circular and each side wall 230 is cylindrical. Other shapes for the walls 228, 230 may be provided. The outer wall surfaces 228a, 230a may match the configuration of the inner wall surfaces 228b, 230b, but the outer wall surfaces 228a, 230a can have any desired configuration, regardless of whether it matches the configuration of the inner wall surfaces 228b, 230b.

The end surfaces 230c of the halves 220, 222 ate together. The recesses 232 align with each other to form the pocket 204 within the housing 202. The half openings 234a, 234b, 234c, 234d align with each other to form the first, second, third and fourth openings 240, 242, 244, 246. In an embodiment, a compressible gasket 247 is provided between the end surfaces 230c of the halves 220, 222. In an embodiment, the gasket 247 is formed of foam or rubber.

In an embodiment, the sensor opening 226 is provided through one of the walls of the second half 222. In an embodiment, the sensor opening 226 is provided through the base wall 228 of the second half 222 and extends from the outer wall surface 228a to the inner wall surface 228b and is in fluid communication with the pocket 204. The sensor opening 226 is separated from the openings 240, 242, 244, 246. The sensor opening 226 may be provided at the center of the base wall 228. The sensor opening 226 is sized and positioned based on the sensor 64 to be used and the desired location of the sensor 64. The sensor 64 is configured to be positioned and held in place within the sensor opening 226 of the housing 202 such that the sensor 64 is configured to detect VOCs within the pocket 204.

As shown in FIGS. 11 and 12, the leak sensor assembly 200 is attached to the second portion 26b of the piping system. The housing 202 is configured to be generally positioned such that the first pipe 28 seats in the first opening 240, the second pipe 30 seats in the second opening 242, and the third pipe 50 seats in the third opening 244. As shown in FIGS. 11 and 12, the fourth opening 246 is not occupied by a pipe. As such, the connector 22 and an end portion of each pipe 28, 30, 50 are within the pocket 204 and are generally encapsulated/enclosed by the housing 202. The first opening 240 may be sized to fit loosely around the first pipe 28 or in close proximity to the first pipe 28; the second opening 242 may be sized to fit loosely around the second pipe 30 or in close proximity to the second pipe 30; the third opening 244 may be sized to fit loosely around the third pipe 50 or in close proximity to the third pipe 50. If a fourth pipe (not shown) was connected to the connector 22, the fourth pipe would extend through the fourth opening 246. If a fourth pipe is provided, the fourth opening 246 may be sized to fit loosely around this fourth pipe or in close proximity to this fourth pipe. If a fourth pipe is not provided, the fourth opening 246 may be closed with a plug 245. Compressible gaskets 248, such as foam or rubber blocks, may be provided around the pipes 28, 30, 50 (and the fourth pipe if provided).

When the housing 202 is attached to the first portion 26a of the piping system, the housing 202 is configured to be generally positioned such that the first pipe 28 seats in the first opening 240, the second pipe 30 seats in the second opening 242, and the stem 46 seats within the third opening 244. As such, a portion of the valve 20 and an end portion of each pipe 28, 30 are within the pocket 204 and are generally encapsulated/enclosed by the housing 202. The first opening 240 may be sized to fit loosely around the first pipe 28 or in close proximity to the first pipe 28; the second opening 242 may be sized to fit loosely around the second pipe 30 or in close proximity to the second pipe 30; and the third opening 244 may be sized to fit loosely around the stem 46 or in close proximity to the stem 46. If a fourth pipe (not shown) was connected to the connector 22, the fourth pipe would extend through the fourth opening 246. If a fourth pipe is provided, the fourth opening 246 may be sized to fit loosely around this fourth pipe or in close proximity to this fourth pipe. If a fourth pipe is not provided, the fourth opening 246 may be closed with a plug 245. Compressible gaskets 248, such as foam or rubber blocks, may be provided around the pipes 28, 30, 50 (and the fourth pipe if provided). Compressible gaskets 248, such as foam or rubber blocks, may be provided around the pipes 28, 30 and the stem 46.

When the housing 202 is attached to the third portion 26c of the piping system, the third and fourth openings 244, 246 are eliminated or closed by suitable means, such as a plug. The housing 202 is configured to be generally positioned such that the first pipe 28 seats in the first opening 240 and the second pipe 30 seats in the second opening 242. As such, the connector 22′ and an end portion of each pipe 28, 30 are within the pocket 204 and are generally encapsulated/enclosed by the housing 202. The first opening 240 may be sized to fit loosely around the first pipe 28 or in close proximity to the first pipe 28; the second opening 242 may be sized to fit loosely around the second pipe 30 or in close proximity to the second pipe 30. If a fourth pipe (not shown) was connected to the connector 22, the fourth pipe would extend through the fourth opening 246. If a fourth pipe is provided, the fourth opening 246 may be sized to fit loosely around this fourth pipe or in close proximity to this fourth pipe. If a fourth pipe is not provided, the fourth opening 246 may be closed with a plug 245. Compressible gaskets 248, such as foam or rubber blocks, may be provided around the pipes 28, 30, 50 (and the fourth pipe if provided). Compressible gaskets 248, such as foam or rubber blocks, may be provided around the pipes 28, 30.

Attention is directed to FIGS. 17-21 and a third embodiment of the leak sensor assembly 300 of the present disclosure. While this embodiment is shown with the first portion 26a of the piping system shown in FIGS. 2A and 2B, this third embodiment can be used with the second or third portions 26b, 26c of the piping system shown in FIGS. 3A, 3B and 4.

The housing 302 is formed from a wall 306 having an inner wall surface and an outer wall surface, and first, second and third spaced apart openings 340, 342, 344 provided therethrough. In an embodiment, openings 340 and 344 are 90 degrees apart from each other, openings 344 and 342 are 90 degrees apart from each other, and openings 340 and 342 are aligned with each other. The wall 306 defines the pocket 304 therein into which VOCs accumulate when the housing 302 is attached to the piping system as described herein. A sensor opening 326 is provided through the wall 306, and extends from an inner wall surface to the outer wall surface of the wall 306 and is in fluid communication with the pocket 304. The sensor 64 is positioned and held in place within the sensor opening 326 of the housing 302 such that the sensor 64 is configured to detect VOCs within the pocket 304.

The wall 306 is formed of flexible, breathable fabric, for example, a fabric sold under the registered trademark TYVEK, a non-woven fabric made of high density polyethylene fibers, or fabrics sold under the registered trademarks TYPAR and CERTAWRAP, which are fabrics made of spunbond polypropylene fibers, or fabrics made of polyvinyl chloride (PVC) or polytetrafluoroethylene (PTFE), such as a flexible fabric material sold under the registered trademark TEFLON, or fabrics made of other fluorinated hydrocarbon polymers. The material forming the wall 306 is preferably: (1) microporous, such that it is gas permeable, but not liquid permeable; (2) hydrophobic, such that it tends to repel water; (3) water-proof; (4) weather-proof/weather-resistant (resistant to the formation of cracks, deformation, etc. upon prolonged exposure to the elements); (5) chemically stable, such that it is chemically resistant to organic compounds; (6) anti-tear; and (7) able to withstand a wide temperature range, for example, −40° Celsius to 200° Celsius.

In an embodiment, the housing 302 is formed of two halves 320, 322 which are joined together by suitable securing means 324. The two halves 320, 322 could be hinged/flexible such that the two halves 320, 322 are joined or integrally formed at line 325 which extends from opening 340 to opening 342, but the two halves 320, 322 are separated along the remainder of the intersection between the two halves 320, 322; such the two halves 320, 322 can open and dose similar to a clam shell along line 325, and such that when closed the two halves 320, 322 are further joined together by the securing means 324. That is, the housing 302 is formed of a single part which can be opened or dosed. The two halves 320, 322 can be completely separate from each other and joined together by the securing means 324. The two halves 320, 322 can be secured to one another using any suitable securing means 324, including, but not limited to, fasteners, latches (as shown), tape, hook-and-loop fasteners (such as those sold under the registered trademark VELCRO), magnets, snaps, buttons, etc. The suitable securing means 324 are also preferably ones that allow for the housing 302 to be removed and reinstalled as desired. In other words, the housing 302 is preferably reusable.

Each half 320, 322 includes a cup-shaped wall 328 forming a recess 332 within each half 320, 322. The cup-shaped wall 328 has an outer wall surface 328a, an inner wall surface 328b and an end surface 328c. The cup-shaped wall 328 forms three half openings 334a, 334b, 334c which are sized to approximate the shapes of the pipes 28, 30, 50 and/or the stem 46. In an embodiment, the half openings 334a, 334b, 334c are half circles. The outer wall surfaces 328a may match the configuration of the inner wall surfaces 328b, but the outer wall surfaces 328a can have any desired configuration, regardless of whether it matches the configuration of the inner wall surfaces 328b.

In an embodiment, the sensor opening 326 is provided through the wall 328 of the second half 322 and extends from the outer wall surface 328a to the inner wall surface 328b such that the sensor opening 326 is in fluid communication with the pocket 304. The sensor opening 326 is separated from the openings 340, 342, 344. The sensor opening 326 may be provided at the center of the wall 328. The sensor opening 326 is sized and positioned based on the sensor 64 to be used and the desired location of the sensor 64. The sensor 64 is configured to be positioned and held in place within the sensor opening 326 of the housing 302 such that the sensor 64 is configured to detect VOCs within the pocket 304.

The halves 320, 322 are wrapped around the piping system and the end surfaces 328c of the halves 320, 322 mate together. The halves 320, 322 are secured together by the securing means 324. The recesses 332 align with each other to form the pocket 304 within the housing 302. The half openings 334a, 334b, 334c align with each other to form the first, second and third openings 340, 342, 344. In an embodiment, a compressible gasket (not shown) provided between the end surfaces 328c of the halves 320, 322. When the two cup-shaped halves 320, 322 are mated, a generally ovoid shape is formed for the pocket 304. Other shapes for the halves 320, 322 may be provided.

As shown in FIG. 17, the leak sensor assembly 300 is attached to the first portion 26a of the piping system shown in FIGS. 2A and 2B. The housing 302 is configured to be generally positioned such that the first pipe 28 seats in the first opening 340, the second pipe 30 seats in the second opening 342, and the stem 46 seats in the third opening 344. As such, the valve 20 and an end portion of each pipe 28, 30 are within the pocket 304 and are generally enclosed by the housing 302. The first opening 340 may be sized to fit loosely around the first pipe 28 or in close proximity to the first pipe 28; the second opening 342 may be sized to fit loosely around the second pipe 30 or in close proximity to the second pipe 30; the third opening 344 may be sized to fit loosely around the stem 46 or in close proximity to the stem 46. Compressible gaskets, such as foam or rubber blocks, may be provided around the pipes 28, 30 and the stem 46.

When the housing 302 is attached to the second portion 26b of the piping system, the housing 302 is configured to be generally positioned such that the first pipe 28 seats in the first opening 340, the second pipe 30 seats in the second opening 342, and the third pipe 50 seats within the third opening 344. As such, the connector 2.2 and an end portion of each pipe 28, 30, 50 are within the pocket 304 and are generally encapsulated/enclosed by the housing 302. The first opening 340 may be sized to fit loosely around the first pipe 28 or in close proximity to the first pipe 28; the second opening 342 may be sized to fit loosely around the second pipe 30 or in close proximity to the second pipe 30; and the third opening 344 may be sized to fit loosely around the third pipe 50 or in close proximity to the third pipe 50. Compressible gaskets 348, such as foam or rubber blocks, may be provided around the pipes 28, 30, 50.

When the housing 302 is attached to the third portion 26c of the piping system, the third and fourth openings 244, 246 are eliminated or closed by suitable means, such as a plug. The housing 302 is configured to be generally positioned such that the first pipe 28 seats in the first opening 340 and the second pipe 30 seats in the second opening 342. As such, the connector 22′ and an end portion of each pipe 28, 30 are within the pocket 304 and are generally encapsulated/enclosed by the housing 302. The first opening 340 may be sized to fit loosely around the first pipe 28 or in close proximity to the first pipe 28; the second opening 342 may be sized to fit loosely around the second pipe 30 or in close proximity to the second pipe 30. Compressible gaskets 348, such as foam or rubber blocks, may be provided around the pipes 28, 30.

In an embodiment, the wall 306 of the housing 302 has a fourth opening (not shown) provided therethrough to accommodate a fourth pipe (not shown). If the fourth opening is provided and the fourth pipe is not provided, the fourth opening may be closed with a plug (not shown).

Attention is directed to FIG. 22 and a fourth embodiment of the leak sensor assembly 400 of the present disclosure. While this embodiment is shown with the first portion 26a of the piping system shown in FIGS. 2A and 2B, this fourth embodiment can be used with the second or third portions 26b, 26c of the piping system shown in FIGS. 3A, 3B and 4.

The housing 402 is formed from a wall 406 having first and second ends, and an inner wall surface 412 and an outer wall surface 414 extending between the ends. A first opening 416 is provided through the first end of the wall 406 and a second opening 418 is provided through the second end of the wall 406. The wall 406 defines the pocket 404 therein into which VOCs accumulate when the housing 402 is attached to the piping system as described herein. A sensor opening (not shown) is provided through the wall 406, and extends from an inner wall surface to the outer wall surface of the wall 406 and is in fluid communication with the pocket 404. The sensor 64 is positioned and held in place within the sensor opening of the housing 402 such that the sensor 64 is configured to detect VOCs within the pocket 404.

The wall 406 is formed of flexible, breathable fabric, for example, a fabric sold under the registered trademark TYVEK, a non-woven fabric made of high density polyethylene fibers, or fabrics sold under the registered trademarks TYPAR and CERTAWRAP, which are fabrics made of spunbond polypropylene fibers, or fabrics made of polyvinyl chloride (PVC) or polytetrafluoroethylene (PUT), such as a flexible fabric material sold under the registered trademark TEFLON, or fabrics made of other fluorinated hydrocarbon polymers. The material forming the wall 406 is preferably: (1) microporous, such that it is gas permeable, but not liquid permeable; (2) hydrophobic, such that it tends to repel water; (3) water-proof; (4) weather-proof/weather-resistant (resistant to the formation of cracks, deformation, etc. upon prolonged exposure to the elements); (5) chemically stable, such that it is chemically resistant to organic compounds; (6) anti-tear; and (7) able to withstand a wide temperature range, for example, −40° Celsius to 200° Celsius.

In an embodiment, the inner wall surface 412 is circular in cross-section, yet conical in configuration from the first opening 416 to the second opening 418. In an embodiment, the outer wall surface 414 matches the configuration of the inner wall surface 412, but the outer wall surface 414 can have any desired configuration, regardless of whether it matches the configuration of the inner wall surface 412.

In an embodiment, the housing 402 is formed of two halves (not shown) which are joined together by suitable securing means (not shown). The two halves could be hinged/flexible such that the two halves are joined or integrally formed at line (not shown) which extends from opening 416 to opening 418, but the two halves are separated along the remainder of the intersection between the two halves; such the two halves can open and close similar to a clam shell along line, and such that when closed the two halves are further joined together by the securing means. That is, the housing 402 is formed of a single part which can be opened or closed. The two halves can be completely separate from each other and joined together by the securing means. The two halves can be secured to one another using any suitable securing means, including, but not limited to, fasteners, latches (as shown), tape, hook-and-loop fasteners (such as those sold under the registered trademark VELCRO), magnets, snaps, buttons, etc. The suitable securing means are also preferably ones that allow for the housing 402 to be removed and reinstalled as desired. In other words, the housing 402 is preferably reusable. In an embodiment, the sensor opening is provided through the wall 406 and extends from the inner wall surface 412 to the outer wall surface 414 and is in fluid communication with the pocket 404. The sensor opening is separated from the first and second openings 416, 418. The sensor opening may be provided at a midpoint of the wall 406 between the first and second openings 416, 418. The sensor opening is sized and positioned based on the sensor 64 to be used and the desired location of the sensor 64. The sensor 64 is configured to be positioned and held in place within the sensor opening of the housing 402 such that the sensor 64 is configured to detect VOCs within the pocket 404.

As shown in FIG. 22, the leak sensor assembly 400 is attached to the first portion 26a of the piping system. The housing 402 is configured to be generally positioned such that a portion of the valve 20 seats within the pocket 404. In an embodiment, the stem 46 seats within the first opening 416 and the bonnet 44 and at least a portion of the body 32 seats within the second opening 418 such that the stem 46, the connection between the stem 46 and the bonnet 44, the bonnet 44, and the connection between the bonnet 44 and the body 32 are generally enclosed by the housing 402. The first opening 416 may be sized to fit loosely around the stem 46 or in close proximity to the stern 46; the second opening 418 may be sized to fit loosely around the body 32 or in close proximity to the body 32.

When the housing 402 is attached to the second portion 26b of the piping system, the housing 402 is configured to be generally positioned such that a portion of one of the pipes 28, 30, 50 and a portion of the connector 2.2 seats within the pocket 404. In an embodiment, the pipe 28, 30 or 50 seats within the first opening 416 and the body 52 of the connector 22 seats within the second opening 418 such that the connection between the pipe 28, 30 or 50 and the body 52 are generally enclosed by the housing 402. The first opening 416 may be sized to fit loosely around the pipe 28, 30 or 50 or in close proximity to the pipe 28, 30 or 50; the second opening 418 may be sized to fit loosely around the body or in close proximity to the body 52.

When the housing 402 is attached to the third portion 26c of the piping system, the housing 402 is configured to be generally positioned such that the connector 22′ seats within the pocket 404. In an embodiment, the first pipe 28 seats within the first opening 416 and the connector 22′ seats within the pocket 404, and the second pipe 30 seats within the second opening 418 such that the connections between the connector 22′ and the pipes 28, 30 are generally enclosed by the housing 402. The first opening 416 may be sized to fit loosely around the first pipe 28 or in close proximity to the first pipe 28; the second opening 418 may be sized to fit loosely around the second pipe 30 or in close proximity to the second pipe 30. In an embodiment, the first pipe 28 seats within the first opening 416 and the connector 22′ seats within the pocket 404, and the body 52′ of the connector 22′ seats within the second opening 418 such that the connections between the connector 22′ and the pipe 28 are generally enclosed by the housing 402. The first opening 416 may be sized to fit loosely around the first pipe 28 or in close proximity to the first pipe 28; the second opening 418 may be sized to fit loosely around the body 52′ of the connector 22′ or in close proximity to the body 52′ of the connector 22′.

In an embodiment, the inner wall 414 of the housing 402 is circular in cross-section, yet conical in configuration from the first end of the housing 402 to a middle portion of the housing 402, and conical in configuration from the middle portion of the housing 402 to the second end of the housing 402. A multi-conical shape aids in limiting moisture accumulation in the pocket 404. Of course, it is to be understood that while the inner wall 414 is identified as being multi-conical in shape, the inner wall 414 could take on any other desired shape, or any other shape that is dictated by the configuration of the component, e.g., the valve 20, so as to maintain the desired volume of the pocket 404. For example, in an embodiment, the inner wall 414 of the housing 402 is spherical.

FIG. 23 shows that the housing 402 includes a framework 450 which is preferably rigid in configuration. The framework 450 may be connected to a component of the piping system, such as the stein 46 of the valve 20 or the third pipe 50. As illustrated in FIG. 19, the framework 450 may include a plurality of ribs which extend radially outwardly from the component, such as stem 46, a predetermined distance. The housing 402 is wrapped around the framework 450 to form the pocket 404. The framework 450 may be eliminated.

In an alternative embodiment of the leak sensor assemblies 100, 200, 300, 400, a fan and/or pump 80, see FIG. 23, could be provided in the housings 102, 202, 302, 402. If a fan is provided, the fan may be at a position generally opposite the sensor(s) 64, such that the fan would blow/direct any leaked gases within the pockets 104, 204, 304, 404 toward the sensors 64, thereby aiding in ensuring that the sensors 64 would be able to detect the presence and concentration level of the leaked gas. If a pump is provided, the pump would suck/direct any leaked gases within the pockets 104, 204, 304, 404 toward the sensors 64, thereby aiding in ensuring that the sensors 64 would be able to detect the presence and concentration level of the leaked gas.

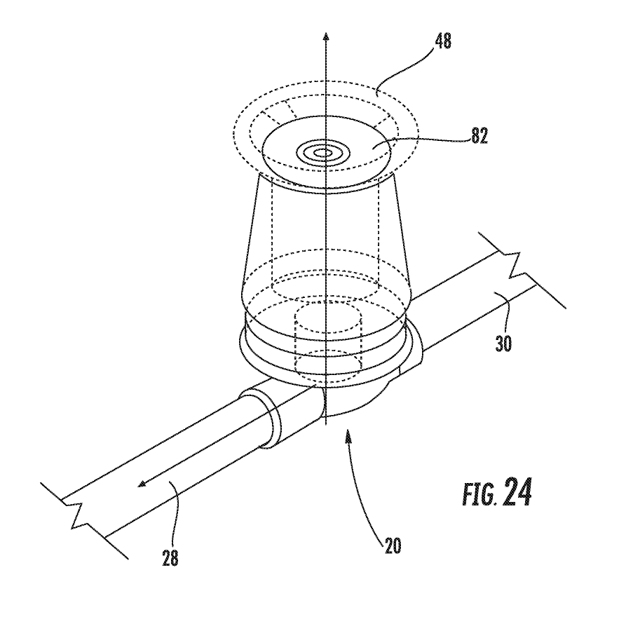

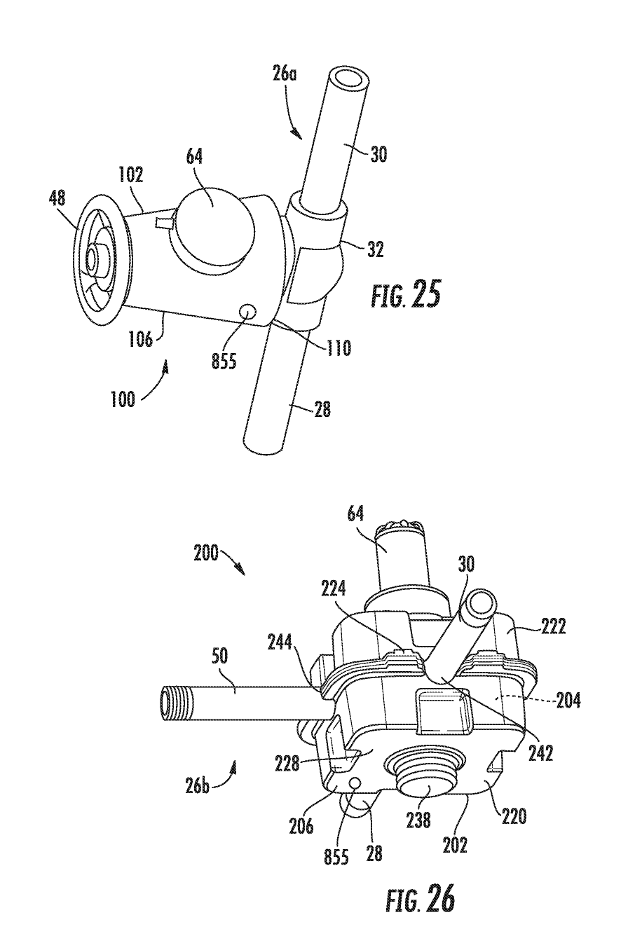

In yet another alternative embodiment, the leak sensor assemblies 100, 200, 300, 400 could be provided with a hydrophobic/gas permeable filler 82 in one or more portions of the pockets 104, 204, 304, 404. For instance, as illustrated in FIG. 24, which generally depicts the leak sensor assembly 100, the filler 82 is provided in the pocket 104 at or proximate to the first end 116 of the housing 102. The provision of the filler 82 thus minimizes the effect of wind in the pocket 104 which could affect the ability of the sensor 64 to properly sense/detect the presence of a VOC therein. One or more fillers 82 could be provided in the pockets 104, 204, 304, 404, and the thickness of the fillers 82 can be varied/optimized. The filler 82 may further completely or substantially fill the pockets 104, 204, 304, 404. As the filler 82 is water/gas permeable, it should not interfere with the operation of the sensors 64 and/or the overall effectiveness of the leak sensor assemblies 100, 200, 300, 400. The filler 82 may also have the advantage of providing further stability in positioning of the housing 102, 202, 302, 402 relative to the component, thus better ensuring that the desired volume of the pockets 104, 204, 304, 404 is maintained.

The reasons for providing the housing 102, 202, 302, 402 with openings 116, 118, 240, 242, 244, 246, 340, 342, 344, 416, 418 thereof are threefold. First, upon a leak of VOCs occurring at the valve 20/connector 22, 22′, the VOCs are provided in the pocket 104, 204, 304, 404. However, as it is not desirable for the concentration of the VOCs to build-up in the pocket 104, 204, 304, 404, the openings 116, 118, 240, 242, 244, 246, 340, 342, 344, 416, 418 provide outlets which reduce VOC accumulation. Second, the openings 116, 118, 240, 242, 244, 246, 340, 342, 344, 416, 418 allow for any moisture formed in the pocket 104, 204, 304, 404 to be drained therefrom. In this regard, the sizes of the openings 116, 118, 240, 242, 244, 246, 340, 342, 344, 416, 418 should be optimized to minimize the effect of weather conditions on sensor 64 reading while ensuring no moisture build-up inside the housing 102, 202, 302, 402. If desired, one or both of the openings 116, 118, 240, 242, 244, 246, 340, 342, 344, 416, 418 could be closed off, so long as the closing off of these openings 116, 118, 240, 242, 244, 246, 340, 342, 344, 416, 418 does not have any negative effects on the operation of the sensor 64 and/or the safety of the process plant. Also, if desired, the openings 116, 118, 240, 242, 244, 246, 340, 342, 344, 416, 418 may be sealed with the stem 46/body 32/body 52, 52′/pipes 28, 30, 50 and the openings 116, 118, 240, 242, 244, 246, 340, 342, 344, 416, 418 could be provided as one or more apertures through the housing 102, 202, 302, 402 from the inner wall surface to the outer wall surface. It should also be understood that as the fabric forming the walls 306, 406 of the housings 302, 402 is preferably a microporous fabric, the microporous fabric would have a multitude of microscopic openings that would act as drain openings for preventing the build-up of VOCs and/or moisture within the housings 302, 402. Third, the housings 102, 202, 302, 402 will act to reduce any bulk water and/or bulk air flow (e.g., wind) from entering the pockets 104, 204, 304, 404 and disrupting the operation of the sensor 64.

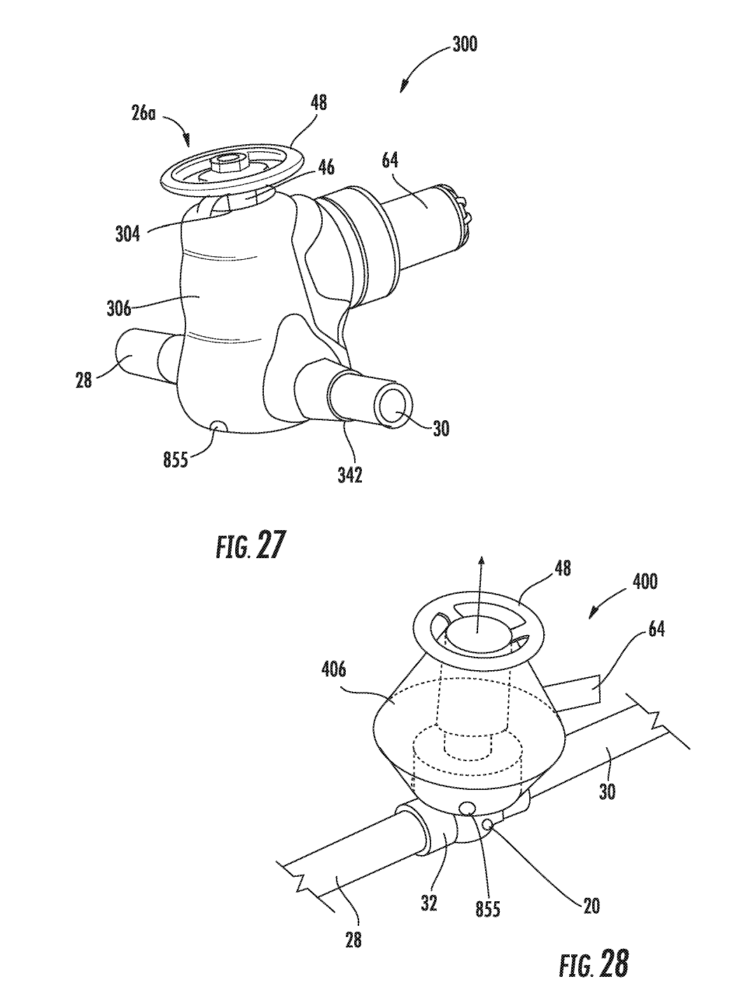

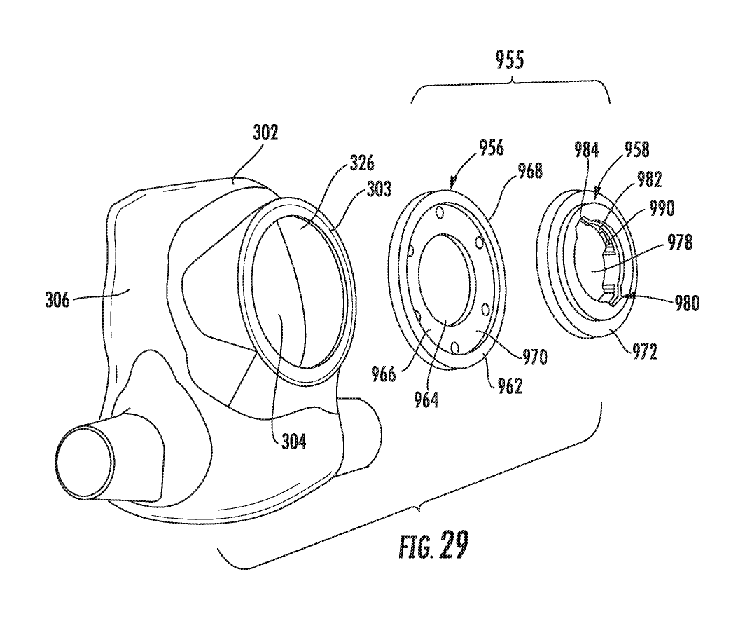

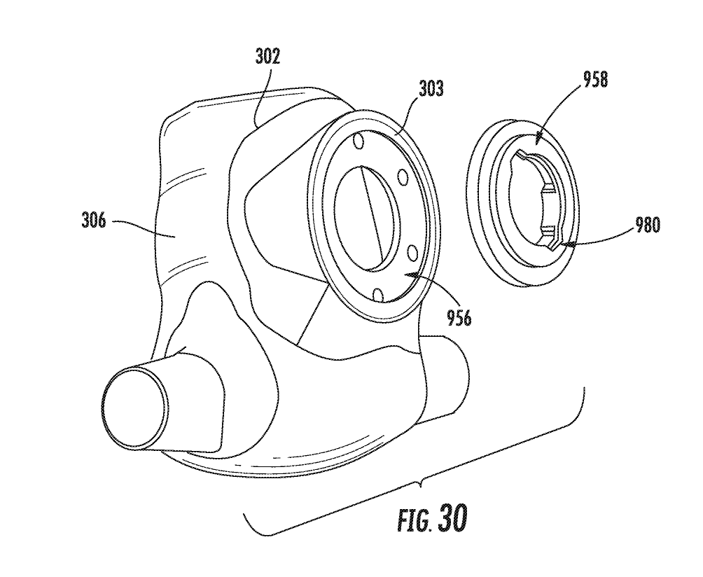

In any of the embodiments, at least one drain opening 855 is preferably provided in the housing 102, 202, 302, 402 and is preferably provided in a position which is closest or substantially closest to the ground, but which is away from the openings 116, 118, 240, 242, 244, 246, 340, 342, 344, 416, 418. That is, the one or more drain openings 855 is provided along a lower portion of the housing 102, 202, 302, 402 as this is the position which is closest or substantially closest to the ground. The one or more drain openings 855 is in fluid communication with the pocket 102, 202, 302, 402. The position of the one or more drain openings 855 will vary depending upon the orientation of the housing 102, 202, 302, 402. For example, when the leak sensor assembly 100 is in the position shown in FIG. 25, the one or more drain openings 855 is preferably provided in the wall 102 and may be proximate the second end 110 of the wall 106. For example, when the leak sensor assembly 200 is in the position shown in FIG. 26, the one or more drain openings 855 is preferably provided in the base wall 228 of the housing 202. For example, when the leak sensor assembly 300 is in the position shown in FIG. 27, the one or more drain openings 855 is preferably provided in the wall 306 underneath the connection to the pipes 28, 30. For example, when the leak sensor assembly 400 is in the position shown in FIG. 28, the one or more drain openings 855 is preferably provided in the wall 406 proximate to the second end and above the pipes 28, 30. The one or more drain openings 855 aids/assists in allowing any moisture in the pocket 104, 204, 304, 404 to exit the pocket 104, 204, 304, 404.

In some embodiments, the sensor 64 is permanently mounted to the housing 102, 202, 302, 402 with the sensor 64 is in fluid communication with the pocket 104, 204, 304, 404 formed by the housing 102, 202, 302, 402. By permanently mounted, this means that the sensor 64 is not readily removable from the housing 102, 202, 302, 402. In an embodiment, the sensor 64 is permanently attached to the housing 102, 202, 302, 402 by suitable means, such as adhesives, etc.

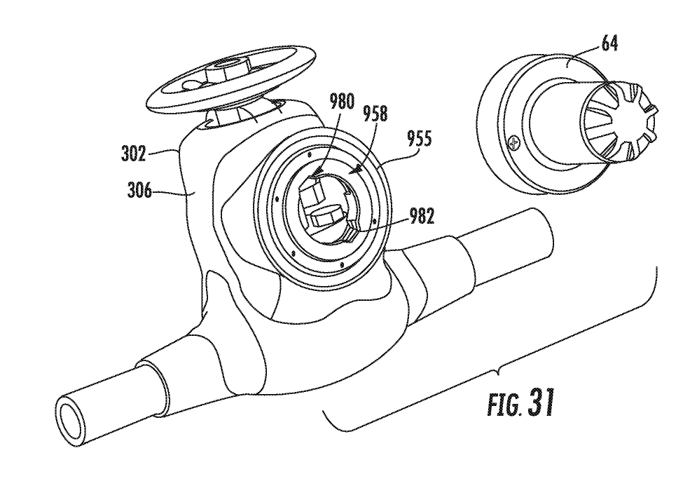

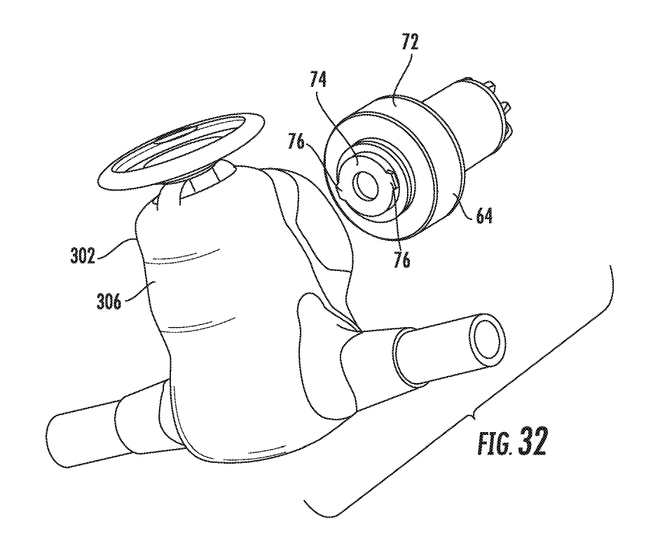

In an embodiment, the sensor 64 is removably mounted to the housing 102, 202, 302, 402 by a collar assembly 955 which forms part of the housing 102, 202, 302, 402 and provides part of the pocket 104, 204, 304, 404. The collar assembly 955 allows for the removable connection of the sensor 64 to the housing 102, 202, 302, 402 such that the sensor 64 can be removed to be repaired/replaced as desired. An example of the collar assembly 955 is shown in FIGS. 29-33 as assembled to the third embodiment of the housing 302.

In an embodiment, the sensor 64 includes a body 72 in which a sensing element is provided. The body 72 of the sensor 64 has a first part 74 of an interengaging assembly provided thereon, as best shown in FIG. 32. In an embodiment, the first part 74 of the interengaging assembly is formed by at least two locking ears 76 provided on the body 72 of the sensor 64. In an embodiment, the locking ears 76 extend outwardly from an end of the body 72 such that a space is provided between the locking ears 76 and the end of the body 72.

The collar assembly 955 includes an inner collar 956 and an outer collar 958 which has a portion 303 of the wall 306 of the housing 302 surrounding the sensor opening 326 sandwiched therebetween.

In an embodiment, the inner collar 956 is formed from a wall 962 having a central aperture 964 therethrough which forms the sensor opening 326. The inner collar 956 may have a base wall 966 and a skirt 968 which extends outwardly from the base wall 966 such that a recess 970 is formed by the inner collar 956.

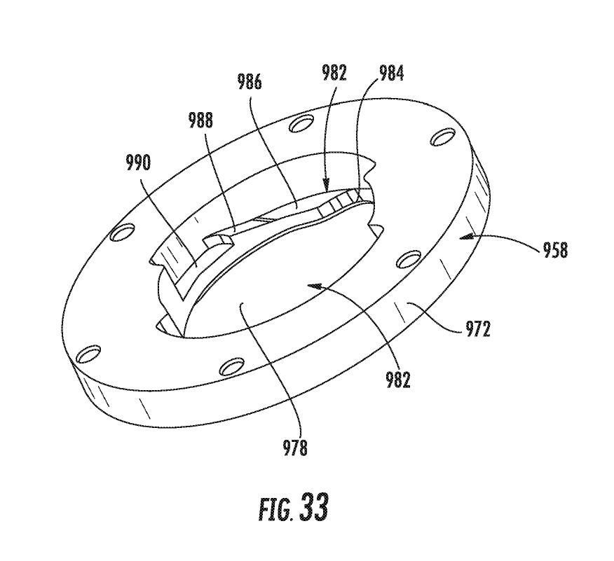

The outer collar 958 is formed of a wall 972 having a central aperture 978 therethrough. The wall 972 of the outer collar 958 has a second, mating part 980 of the interengaging assembly provided thereon. In an embodiment, the second, mating part 980 is formed by at least two recesses 982 provided on the outer collar 958. As best shown in FIG. 33, in an embodiment, each recess 982 has an opening 984 at an end of the wall 972, a planar section 986 extending from the opening 984 to a ramped section 988, and a planar seat section 990 extending from the ramped section 988 which is aligned or generally aligned with the planar section 986.

The outer collar 958 seats within the recess 970 of the inner collar 956 and the portion 303 of the wall 306 of the housing 302 is trapped between the ring walls 962, 972 of the inner and outer collars 956, 958. The inner and outer collars 956, 958 are suitably attached to each other in a known manner, such as by fasteners which extend through the inner and outer collars 956, 958 and the housing 302.

The sensor 64 is removably mounted to the housing 302/955. To attach the sensor 64 to the housing 302/955, the locking ears 76 are inserted into the openings 984 of the recesses 982 until the locking ears 76 move past the planar sections 986 and the wall forming the planar sections 986 are aligned with the space between the locking ears 76 and the end of the sensor body 72. Thereafter, the sensor 64 is twisted relative to the housing 302/955. The locking ears 76 slide along the planar sections 986 and then along the ramped sections 988. The locking ears 76 seats within the planar seat sections 990 such that the wall forming the recesses 982 is trapped between the locking ears 76 and the end of the sensor body 72. This prevents the accidental disengagement of the sensor 64 from the housing 302/955. To detach the sensor 64 from the housing 302/955, the sensor 64 is twisted relative to the housing 302/955 in the opposite direction until the locking ears 76 are released from the recesses 982. In an alternate embodiment, the locking ears 76 are provided on the housing 302/955 and the recesses 982 are provided on the sensor 64.

In alternative embodiments of the leak sensor assemblies 100, 200, 300, 400, the leak sensor assemblies 100, 200, 300, 400 could be provided with a plurality of sensors 64 rather than a single sensor 64.

While the pockets 104, 204, 304, 404 appear in the figures to define a substantial volume, the housing 102, 202, 302, 404 may closely conform to the components of the piping system such that a minimal volume is provided by the pockets 104, 204, 304, 404.

Attention is directed to FIGS. 34-40 and the fifth embodiment of the leak sensor assembly 500 of the present disclosure. While this embodiment is shown with the first portion 26a of the piping system shown in FIGS. 2A and 2B, this fifth embodiment can be used with the second or third portions 26b, 26c of the piping system shown in FIGS. 3A, 3B and 4.

The housing 502 is formed from a wall 506 having first and second ends 508, 510 and a securement 592 attached to the first end 508 of the wall 506. The pocket 504 extends from the first end 508 to a sensor opening 526 at the second end 510. The sensor opening 526 is in fluid communication with the pocket 504. The sensor 64 is positioned and held in place within the sensor opening 526 of the housing 502 such that the sensor 64 is configured to detect VOCs within the pocket 504. The sensor 64 shown in FIG. 32 is used with the housing 502 and the specifics are not repeated herein. In some embodiments, the wall 506 is formed as a ring.

In an embodiment, the wall 506 is formed of a rigid material, such as metal, ceramic, thermoplastic (acrylonitrile butadiene styrene (ABS), Noryl, polyethylene, polypropylene, polyvinyl chloride (PVC), polycarbonate, etc.), and particularly high temperature plastics such as liquid crystal polymer (LCP), syndiotactic polystyrene (SPS), polyvinylidene fluoride (PVDF), nylon, and durable high-performance polyimide-based plastics, such as those sold under the registered trademark VESPEL. The material of the wall 506 is also preferably water-proof, weather-proof (e.g., resistant to the formation of cracks, deformation, etc. upon prolonged exposure to the elements), and chemically resistant to organic compounds. The material of the housing 502 can further desirably withstand a wide temperature range, for example, −40° Celsius to 200° Celsius.

The wall 506 of the housing 502 has a second, mating part 580 of the interengaging assembly provided thereon. In an embodiment, the second, mating part 580 is formed by at least two recesses 582 provided on the housing 502. As best shown in FIG. 36 and 37, in an embodiment, each recess 582 has an opening 584 at the second end 510 of the wall 506, a planar section 586 extending from the opening 584 to a ramped section 588, and a planar seat section 590 extending from the ramped section 588 which is aligned or generally aligned with the planar section 586.

The sensor 64 is removably mounted to the housing 502. To attach the sensor 64 to the housing 502, the locking ears 76 are inserted into the openings 584 of the recesses 582 until the locking ears 76 move past the planar sections 586 and the wall forming the planar sections 586 are aligned with the space between the locking ears 76 and the end of the sensor body 72. Thereafter, the sensor 64 is twisted relative to the housing 502. The locking ears 76 slide along the planar sections 586 and then along the ramped sections 588. The locking ears 76 seats within the planar seat sections 590 such that the wall forming the recesses 582 is trapped between the locking ears 76 and the end of the sensor body 72. This prevents the accidental disengagement of the sensor 64 from the housing 502. To detach the sensor 64 from the housing 502, the sensor 64 is twisted relative to the housing 502 in the opposite direction until the locking ears 76 are released from the recesses 582. In an alternate embodiment, the locking ears 76 are provided on the housing 502 and the recesses 582 are provided on the sensor 64.

In an embodiment as shown in FIG. 34-38, the securement 592 is formed by one or more magnets 594 which are mounted on the wall 506. In an embodiment, a plurality of spaced apart magnets 594 are mounted on the wall 506. In an embodiment, a single magnet 594 extends continuously around the wall 506. In an embodiment, the magnet(s) 594 are attached to the wall 506 by fasteners 596. The wall 506 and the attached sensor 64 are magnetically attached by the magnet(s) 594 to the first, second or third portions 26a, 26b, 26c of the piping system proximate to the components to be monitored, such as the valve 20 or the connector 22, 22′. This positions the wall 506 proximate to the components to be monitored, such as the valve 20 or the connector 22, 22′. The wall 506 and the attached sensor 64 can be released from attachment to the first, second or third portions 26a, 26b, 26c of the piping system by pulling the wall 506, its securement 592 and the attached sensor 64 away from the first, second or third portions 26a, 26b, 26c of the piping system.

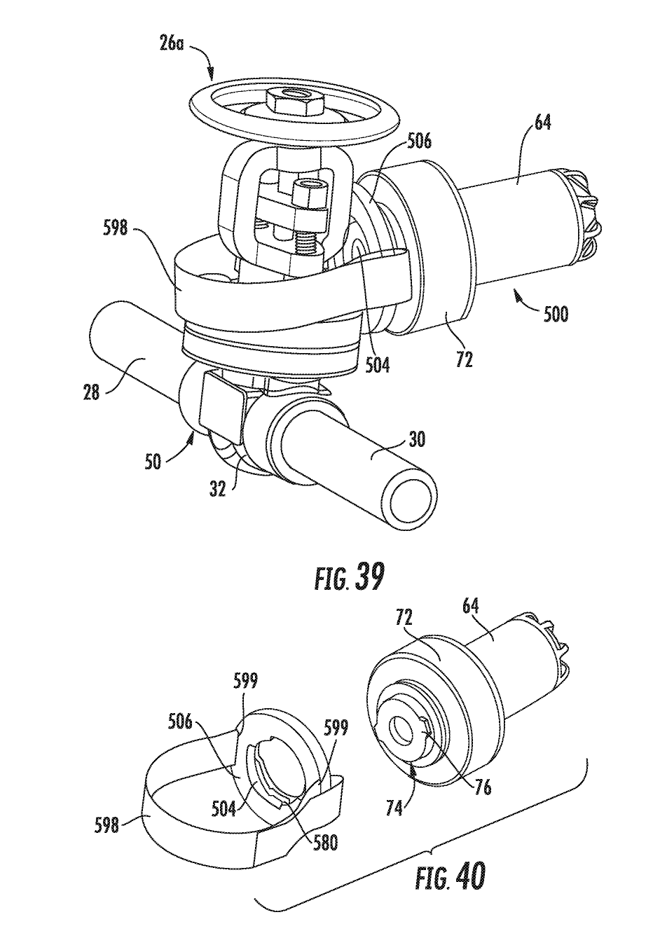

In an embodiment as shown in FIGS. 39 and 40, the securement 592 is formed by a strap 598 which extends through at least one opening 599 in the wall 506. The straps 598 and at least one opening 599 replace the magnets) 594 and fasteners 596.

In an embodiment as shown, two openings 599 are provided and the openings 599 are diametrically opposed. The strap 598 extends through both openings 599 and is wrapped around the first, second or third portions 26a, 26b, 26c of the piping system. This positions the wall 506 proximate to the components to be monitored, such as the valve 20 or the connector 22, 22′. The ends of the strap 598 can be secured to one another or the free end can be secured along the length of the strap 598 by suitable means, such as fasteners, latches, tape, hook-and-loop fasteners (such as those sold under the registered trademark VELCRO), magnets, snaps, buttons, etc. The wall 506, its securement 592 and the attached sensor 64 can be released from attachment to the first, second or third portions 26a, 26b, 26c of the piping system by disengaging the strap 598 and pulling the wall 506 and the attached sensor 64 away from the first, second or third portions 26a, 26b, 26c of the piping system.

In an embodiment, one opening 599 is provided. One end of the strap 598 is permanently attached to the wall 506. The strap 598 is wrapped around the first, second or third portions 26a, 26b, 26c of the piping system and through the opening 599. This positions the wall 506 proximate to the components to be monitored, such as the valve 20 or the connector 22, 22′. The free end can be secured along the length of the strap 598 by suitable means, such as fasteners, latches, tape, hook-and-loop fasteners (such as those sold under the registered trademark VELCRO), magnets, snaps, buttons, etc. The wall 506, its securement 592 and the attached sensor 64 can be released from attachment to the first, second or third portions 26a, 26b, 26c of the piping system by disengaging the strap 598 and pulling the wall 506 and the attached sensor 64 away from the first, second or third portions 26a, 26b, 26c of the piping system.

While the sensor 64 has been shown and described as being removably mounted to the housing 502, in some embodiments, the sensor 64 is permanently mounted to the housing 502 with the sensor 64 in fluid communication with the pocket 504 formed by the housing 502. By permanently mounted, this means that the sensor 64 is not readily removable from the housing 502. In an embodiment, the sensor 64 is permanently attached to the housing 502 by suitable means, such as adhesives, etc.

In an embodiment, the securement 592 could be provided by a combination of the strap 598 and the magnet(s) 594.

Attention is directed to FIG. 41 and the sixth embodiment of the leak sensor assembly 600 of the present disclosure. While FIG. 41 does not show any of the portions 26a, 26b, 26c of the piping system shown in FIGS. 2A-4, this sixth embodiment can be used with the any of the portions 26a, 26b, 26c of the piping system.

As shown in FIG. 41, in an embodiment, the leak sensor assembly 600 includes the leak sensor assembly 500 and a secondary housing 1003. The leak sensor assembly 500 is attached to the secondary housing 1003 which generally surrounds/surrounds the portion 26a, 26b, 26c of the piping system (not shown in FIG. 41 for clarity). The secondary housing 1003 may take the form of any of the housings 102, 202, 302, 402 and forms a secondary pocket 1005 which is in fluid communication with the pocket 504. The leak sensor assembly 500 is attached to the portion 26a, 26b, 26c of the piping system and then the secondary housing 1003 is mounted around the portion 26a, 26b, 26c of the piping system and the secondary housing 1003 is attached to the wall 506 by suitable means, such as adhesive. As shown in FIG. 41, the secondary housing 1003 is in the form of housing 302. In this embodiment of the leak sensor assembly 600, the leak sensor assembly 500 is attached to the portion 26a, 26b, 26c of the piping system by the securement 592 (while the securement 592 of FIGS. 34-38 is shown in FIG. 41, it is to be understood that the securement 592 of FIGS. 39 and 40, or a combination of the securements 592 of FIGS. 34-38 and FIGS. 39 and 40 can be used). In an embodiment of the leak sensor assembly 600 when the secondary housing 1003 takes the form of housing 302, the leak sensor assembly 500 is attached to the portion 26a, 26b, 26c of the piping system and then the secondary housing 1003 is wrapped around the portion 26a, 26b, 26c of the piping system and the secondary housing 1003 is attached to the wall 506 by suitable means, such as adhesive.

Attention is directed to FIG. 42 and the seventh embodiment of the leak sensor assembly 700 of the present disclosure. While this embodiment is shown with the third portion 26c of the piping system shown in FIG. 4, this seventh embodiment can be used with the first or second portions 26a, 26b of the piping system shown in FIGS. 2A-3B. As shown in FIG. 42, in an embodiment, in the seventh embodiment of the leak sensor assembly 700, the leak sensor assembly 500 is attached to the portion 26c of the piping system and the housing 702 is formed by waterproof, flexible tape which is wrapped around the third portion 26c of the piping system which forms the wall 704. When the tape which forms the wall 704 is wrapped around the piping system, openings 716, 718 are formed around the pipes 28, 30. When the tape which forms the wall 704 is wrapped around the leak sensor assembly 700, in an embodiment, the tape which forms the wall 704 is wrapped around the wall 506 and forms an opening 703 around the leak sensor assembly 700.

In each embodiment, the sensor 64 can be provided with power via hard-wiring to main power or via a battery. If the sensor 64 requires significant power consumption, then hard-wiring to main power would be preferred. However, when operated on battery power, the sensor 64 may be turned on to perform a gas measurement intermittently, such as at a fixed interval, e.g. every month, every week, every day, every hour, etc. A battery-powered sensor 64 may be provided with or without a solar panel. A non-dispersive infrared sensor 64, for example, has a start-up time of no more than 1 minute. This means the sensor 64 only needs to operate less than 30 minutes each year, assuming the sensor 64 is configured to take a gas reading once per month. Even with 200 mW power consumption, one CR2032 lithium battery that has 230 mAh capacity can easily last over 5 years.

In each embodiment, since the sensor 64 is attached to the housing 102, 202, 302, 402, 502, 702 the sensor 64 is not readily affected by outdoor environmental conditions, such as wind, rain, and temperature variations. Due to the provision of the housing 102, 202, 302, 402, 502, 702 around the valve 20/connector 22, 22′, leakage of VOCs out of the valve 20/connector 22, 22′ is held within the pocket 104, 204, 304, 404, 504 such that gas concentration within the pocket 104, 204, 304, 404, 504 becomes generally uniform/less sensitive to the position of the sensor 64. As such, the sensor 64 does not need to be positioned at the exact point where the leak is occurring. Rather, the sensor 64 can be generally positioned at certain locations where sensor 64 readings are most consistent no matter where leaks occur and where effect of the weather is minimal.

The housing 102, 202, 302, 402, 502, 702 acts both as a weather jacket, to prevent the valve 20/connector 22, 22′ and the sensor 64 to exposure of the outdoor environmental conditions, thus avoiding corrosion of the valve 20/connector 22, 22′ and flooding of the sensor 64, and as a gas concentrator, to allow for the diffusion of gas to be evenly distributed/concentrated within the pocket 104, 204, 304, 404, 504. The secondary housing 1003 acts both as a weather jacket, to prevent the valve 20/connector 22, 22′ and the sensor 64 to exposure of the outdoor environmental conditions, thus avoiding corrosion of the valve 20/connector 22, 22′ and flooding of the sensor 64, and as a gas concentrator, to allow for the diffusion of gas to be evenly distributed/concentrated within the pocket 504.

In an embodiment, the control unit 68, see FIG. 5, and/or the sensor network 70, see FIG. 6, could also be operatively associated with the handwheels 48 of the valves 20 in order to automatically/remotely control the valves 20 as desired in view of the information from the readings of the sensor 64.

It is to be understood that while the leak sensor assemblies 100, 200, 300, 400, 500, 600, 700 have been described and illustrated for use in connection with detecting leaks at valves 20 and at connectors 22, 22′, that the same principles could also be applied at other components of the piping system, for example, pumps, sampling connections, compressors, pressure-relief devices, and open-ended lines.

While particular embodiments are illustrated in and described with respect to the drawings, it is envisioned that those skilled in the art may devise various modifications without departing from the spirit and scope of the appended claims. It will therefore be appreciated that the scope of the disclosure and the appended claims is not limited to the specific embodiments illustrated in and discussed with respect to the drawings and that modifications and other embodiments are intended to be included within the scope of the disclosure and appended drawings. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the disclosure and the appended claims.

We claim:

1. A leak sensor assembly, the leak sensor assembly configured to detect the leakage of gas from a component of a piping system, the leak sensor assembly comprising:
a housing defining a pocket, at least two component receiving openings which are configured to receive components of the piping system, a sensor opening, and one or more drain openings configured to provide an exit for leaked gas and/or moisture that has built-up within the pocket, the sensor opening being separated from the at least two component receiving openings, the one or more drain openings being separated from the at least two component receiving openings and the sensor opening, the housing being configured to be positioned proximate to the component, wherein gas leaked from the component concentrates within the pocket in a generally uniform dispersion; and
at least one sensor mounted on the housing in the sensor opening, the at least one sensor configured to detect the presence and concentration of gas within the pocket of the housing.

2. The leak sensor assembly as defined in claim 1, wherein the one or more drain openings are provided at a lower portion of the housing.

3. The leak sensor assembly as defined in claim 1, wherein the housing is formed of a microporous material and the one or more drain openings are provided by one or more microscopic openings in the microporous material.

4. The leak sensor assembly as defined in claim 1, wherein the housing has an inner wall surface and an outer wall surface, the inner wall surface is generally conical.

5. (canceled)

6. The leak sensor assembly as defined in claim 1, wherein the housing is formed of a single part which can be opened or closed around the piping system.

7. (canceled)

8. (canceled)

9. (canceled)

10. (canceled)

11. The leak sensor assembly as defined in claim 1, wherein the housing further comprises a collar forming the sensor opening, the collar having an attachment thereon, and the sensor comprises a body having an attachment thereon, wherein the attachment on the collar is releasably engaged with the attachment on the sensor.

12. (canceled)

13. The leak sensor assembly as defined in claim 1, wherein the housing is formed of two halves which are mated together around the piping system, and are attached to each other by a securing mechanism.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The leak sensor assembly as defined in claim 1, wherein the at least one component receiving openings closely conform in shape to the components of the piping system.

30. The leak sensor assembly as defined in claim 1, further comprising a pump which is configured to draw leaked gas within the pocket toward the at least one sensor.

31. The leak sensor assembly as defined in claim 1, further comprising a fan which is configured to blow leaked gas within the pocket toward the at least one sensor.

32. (canceled)

33. (canceled)

34. (canceled)

35. (canceled)

36. (canceled)

37. (canceled)

38. The leak sensor assembly as defined in claim 1, wherein a hydrophobic/gas permeable filler is provided in the pocket.

39. (canceled)

40. A leak sensor assembly, the leak sensor assembly configured to detect the leakage of gas from a component of a piping system, the leak sensor assembly comprising:
a housing defining a pocket, at least two component receiving openings which are configured to receive components of the piping system and a sensor opening, the housing being configured to be positioned proximate to the component, wherein gas leaked from the component concentrates within the pocket in a generally uniform dispersion,
the housing having a first part of an interengaging assembly thereon;
a sensor releasably mounted to the housing in the sensor opening, the sensor comprising a body having a second part of the interengaging assembly thereon, the sensor configured to detect the presence and concentration of gas within the pocket of the housing; and
wherein the first part is releasably engaged with the second part.

41. The leak sensor assembly as defined in claim 40, wherein one of the first part and the second part comprises at least a pair of ears and the other of the first part and the second part comprises at least a pair of passageways into which the ears are received.

42. (canceled)

43. The leak sensor assembly as defined in claim 40, wherein the housing has a wall and an inner collar attached to the wall, which inner collar further defines the pocket, and the sensor opening is formed by an outer collar attached to the wall, wherein the wall is secured between the inner and outer collars.

44. The leak sensor assembly as defined in claim 43, wherein the inner and outer collars are attached to each other.

45. (canceled)

46. (canceled)

47. (canceled)

48. (canceled)

49. (canceled)

50. A leak sensor assembly, the leak sensor assembly configured to detect the leakage of gas from a component of a piping system, the leak sensor assembly comprising:
a housing having a rigid wall and a securement provided on the wall, the securement is configured to releasably attach the wall to the piping system, the wall defining a pocket, at least two component receiving openings which are configured to receive components of the piping system and a sensor opening in communication with the pocket, the wall being configured to be positioned proximate to the component via the securement, wherein gas leaked from the component concentrates within the pocket in a generally uniform dispersion; and
at least one sensor mounted on the wall in the sensor opening, the at least one sensor configured to detect the presence and concentration of gas within the pocket of the housing.

51. The leak sensor assembly as defined in claim 50, wherein the wall has a first part of an interengaging assembly thereon and the sensor comprises a body having a second part of the interengaging assembly thereon, wherein the first part is releasably engaged with the second part.

52. The leak sensor assembly as defined in claim 51, wherein one of the first part and the second part comprises at least a pair of ears and the other of the first part and the second part comprises at least a pair of passageways into which the ears are received.

53. The leak sensor assembly as defined in claim 50, further comprising a second housing attached to the first housing.

54. (canceled)

55. (canceled)

56. The leak sensor assembly as defined in claim 53, wherein the second housing is attached to the wall.

57. The leak sensor assembly as defined in claim 50, wherein the securement comprises a plurality of magnets mounted on the wall, the magnets being configured for magnetic attachment to the component.

58. The leak sensor assembly as defined in claim 50, wherein the securement comprises a strap attached to the wall, the strap being configured for attachment to the component.

59. (canceled)

60. A leak sensor assembly, the leak sensor assembly configured to detect the leakage of gas from a component of a piping system, the leak sensor assembly comprising:
a housing having a first end, a second opposite end, an inner wall surface extending from the first end to the second end, an outer wall surface extending from the first end to the second end, the housing defining at least two component receiving openings which are configured to receive components of the piping system and a sensor opening extending from the inner wall surface to the outer wall surface, wherein the inner wall surface is generally conical from the first end to the second end and defining a generally conical pocket, the housing being configured to be positioned proximate to the component, wherein gas leaked from the component concentrates within the pocket in a generally uniform dispersion; and
at least one sensor mounted on the housing in the sensor opening, the at least one sensor configured to detect the presence and concentration of gas within the pocket of the housing.

61. (canceled)

62. (canceled)

63. (canceled)
